(19) United States
(12) Patent Application Publication
(10) Pub. No.: US 2011/0071180 A1
(43) Pub. Date: Mar. 24, 2011

(54) SUB-TYPE SELECTIVE AMIDES OF DIAZABICYCLOALKANES

(75) Inventors: Srinivasa Rao Akireddy, Winston-Salem, NC (US); Balwinder Singh Bhatti, Winston-Salem, NC (US); Scott R. Breining, Winston-Salem, NC (US); Philip S. Hammond, Pinnacle, NC (US); Ronald Joseph Heemstra, Lewisville, NC (US); Anatoly A. Mazurov, Greensboro, NC (US); Matt S. Melvin, Winston-Slem, NC (US); Lan Miao, Advance, NC (US); V. Srinivasa Murthy, Winston-Salem, NC (US); Jon-Paul Strachan, Burlington, NC (US); Yunde Xiao, Clemmons, NC (US)

(73) Assignee: Targacept, Inc., Winston-Salem, NC (US)

(21) Appl. No.: 12/919,970

(22) PCT Filed: Mar. 4, 2009
(86) PCT No.: PCT/US09/36009

§ 371 Date: Dec. 7, 2010

Related U.S. Application Data
(63) Continuation of application No. 61/033,959, filed on Mar. 5, 2008.

(51) Int. Cl. A61K 31/439; C07D 487/04; C07D 471/08; A61K 31/407; A61K 31/4439; A61P 25/00; A61P 25/28; A61P 25/18

(52) U.S. Cl. 514/278; 548/453; 546/276.7; 546/122; 548/407; 546/15; 514/300; 514/412; 514/409; 514/338

(57) ABSTRACT

The present invention relates to compounds of the following formula (I) that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

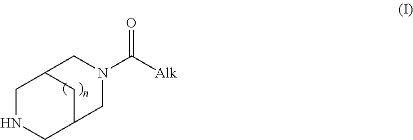

### FIELD OF THE INVENTION

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### BACKGROUND OF THE INVENTION

The therapeutic potential of compounds that target neuronal nicotinic receptors (NNRs), also known as nicotinic acetylcholine receptors (nAChRs), has been the subject of several reviews. See, for example, Breining et al., Ann. Rep. Med. Chem. 40: 3 (2005), Hogg and Bertrand, Curr. Drug Targets: CNS Neural. Disord. 3: 123 (2004), Suto and Zacharias, Expert Opin. Ther. Targets 8: 61 (2004), Dani et al., Bioorg. Med. Chem. Lett. 14: 1837 (2004), Bencherif and Schmitt, Curr. Drug Targets: CNS Neural. Disord. 1: 349 (2002). Among the kinds of indications for which NNR ligands have been proposed as therapies are cognitive disorders, including Alzheimer's disease, attention deficit disorder, and schizophrenia (Newhouse et al., Curr. Opin. Pharmacol. 4: 36 (2004), Levin and Rezvani, Curr. Drug Targets: CNS Neural. Disord. 1: 423 (2002), Graham et al., Curr. Drug Targets: CNS Neural. Disord. 1: 387 (2002), Ripoll et al., Curr. Med. Res. Opin. 20(7): 1057 (2004), and McEvoy and Allen, Curr. Drug Targets: CNS Neural. Disord. 1: 433 (2002)); pain and inflammation (Decker et al., Curr. Top. Med. Chem. 4(3): 369 (2004), Vincler, Expert Opin. Invest. Drugs 14(10): 1191 (2005), Jain, Curr. Opin. Inv. Drugs 5: 76 (2004), Miao et al., Neuroscience 123: 777 (2004)); depression and anxiety (Shytle et al., Mol. Psychiatry 7: 525 (2002), Damaj et al., Mol. Pharmacol. 66: 675 (2004), Shytle et al., Depress. Anxiety 16: 89 (2002)); neurodegeneration (O'Neill et al., Curr. Drug Targets: CNS Neural. Disord. 1: 399 (2002), Takata et al., J. Pharmacol. Exp. Ther. 306: 772 (2003), Marrero et al., J. Pharmacol. Exp. Ther. 309: 16 (2004)); Parkinson's disease (Jonnala and Buccafusco, J. Neurosci. Res. 66: 565 (2001)); addiction (Dwoskin and Crooks, Biochem. Pharmacol. 63: 89 (2002), Coe et al., Bioorg. Med. Chem. Lett. 15(22): 4889 (2005)); obesity (Li et al., Curr. Top. Med. Chem. 3: 899 (2003)); and Tourette's syndrome (Sacco et al., J. Psychopharmacol. 18(4): 457 (2004), Young et al., Clin. Ther. 23(4): 532 (2001)).

A limitation of some nicotinic compounds is that they are associated with various undesirable side effects, for example, by stimulating muscle and ganglionic receptors. There is a need, therefore, to have compounds, compositions and methods for preventing or treating various conditions or disorders, for example CNS disorders, including alleviating the symptoms of these disorders, where the compounds exhibit nicotinic pharmacology with a beneficial effect, for example, upon the functioning of the CNS, preferably without significant associated side effects. Further, there is a need to provide compounds, compositions and methods that affect CNS function without significantly affecting those receptor subtypes which have the potential to induce undesirable side effects, including, for example, appreciable activity at cardiovascular and skeletal muscle sites.

### SUMMARY OF THE INVENTION

The present invention includes a compound of Formula I:

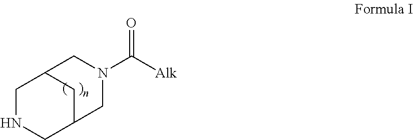

wherein n is 0 or 1; and
Alk is alkyl, cycloalkyl, alkenyl, cycloalkenyl, alkynyl, or cycloalkynyl, each of which may be substituted with one, two, or three of alkyl, substituted alkyl, alkenyl, substituted alkenyl, alkynyl, substituted alkynyl, heterocyclyl, substituted heterocyclyl, cycloalkyl, substituted cycloalkyl, aryl, heteroaryl, substituted aryl, substituted heteroaryl, alkylaryl, alkylheteroaryl, substituted alkylaryl, substituted alkylheteroaryl, arylalkyl, heteroarylalkyl, substituted arylalkyl, substituted heteroarylalkyl, halogen, —OR′, ═O, —NR′R″, haloalkyl, —CN, —NO₂, —SR', —N₃, —C(═O)NR′R″, —NR′C(═O)R″, —C(═O)R′, —C(═O)OR′, —OC(═O)R′, —OC(═O)NR′R″, —NR′C(═O)OR″, —SO₂NR′R″, and —NR′SO₂R″,
where R′ and R″ are independently selected from hydrogen, alkyl, cycloalkyl, heterocyclyl, aryl, heteroaryl, or arylalkyl,
or R′ and R″ and the atoms to which they are attached together can form a three- to eight-membered heterocyclic ring,
wherein the term substituted, as applied to alkyl, alkenyl, alkynyl, heterocyclyl, cycloalkyl, aryl, heteroaryl, alkylaryl, alkylheteroaryl, arylalkyl, and heteroarylalkyl, refers to substitution with one or more alkyl, aryl, heteroaryl, halogen, —OR′, or —NR′R″ groups, where R′ and R″ are as defined;
or a pharmaceutically acceptable salt thereof.

One embodiment of the present invention provides amide compounds which can be formed from certain aliphatic carboxylic acids and certain diazabicycloalkanes, particularly 3,7-diazabicyclo[3.3.0]octane and 3,7-diazabicyclo[3.3.1]nonane aliphatic amides and pharmaceutically acceptable salts thereof. The amide compounds of the present invention bind with high affinity to NNRs of the α4β2 subtype, found in the CNS, and exhibit selectivity for the α4β2 subtype over the α7 NNR subtype, also found in the CNS. The present invention also relates to pharmaceutically acceptable salts prepared from these compounds.

The present invention includes pharmaceutical compositions comprising an amide compound of the present invention or a pharmaceutically acceptable salt thereof. The pharmaceutical compositions of the present invention can be used for treating or preventing a wide variety of conditions or disorders, and particularly those disorders characterized by dysfunction of nicotinic cholinergic neurotransmission or the degeneration of the nicotinic cholinergic neurons.

The present invention includes a method for treating or preventing disorders and dysfunctions, such as CNS disorders and dysfunctions, and also for treating or preventing certain conditions, for example, alleviating pain and inflammation, in mammals in need of such treatment. The methods involve administering to a subject a therapeutically effective amount of an amide compound of the present invention, including a salt thereof, or a pharmaceutical composition that includes such compounds.

More specifically, the present invention includes a method for the treatment or prevention of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, schizophreniform disorder, schizoaffective disorder, cognitive deficit in schizophrenia, and cognitive dysfunction in schizophrenia.

Still further specifically, the present invention includes a method for the treatment or prevention of mild to moderate dementia of the Alzheimer's type, attention deficit disorder, attention deficit hyperactivity disorder, mild cognitive impairment, age-associated memory impairment, cognitive deficit in schizophrenia, and cognitive dysfunction in schizophrenia.

The foregoing and other aspects of the present invention are explained in further detail in the detailed description and examples set forth below.

### BRIEF DESCRIPTION OF THE FIGURES

FIG. 1 is a chart showing the results of a study on object recognition in rats treated orally with N-(propanoyl)-3,7-diazabicyclo[3.3.0]octane. The results are shown as a function of recognition index (%) versus dose (mg/kg). N-(Propanoyl)-3,7-diazabicyclo[3.3.0]octane is active orally in rats at 0.3 mg/kg in novel object recognition (NOR) task.

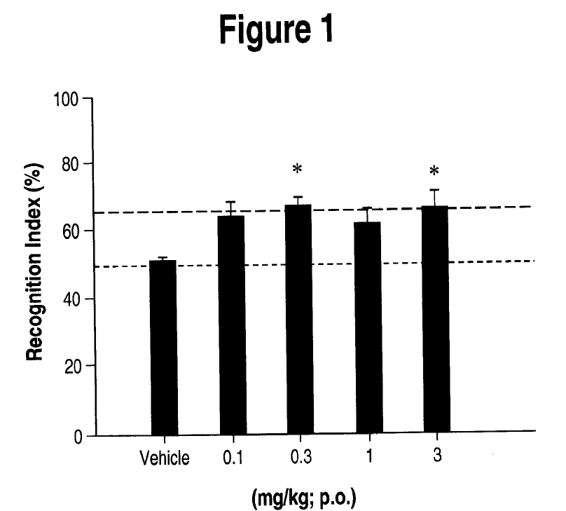

### DETAILED DESCRIPTION

The following definitions are meant to clarify, but not limit, the terms defined. If a particular term used herein is not specifically defined, such term should not be considered indefinite. Rather, terms are used within their accepted meanings.

As used herein the term “alkyl” refers to a straight or branched chain hydrocarbon having one to twelve carbon atoms, preferably one to eight carbon atoms, which may be optionally substituted as herein further described, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well. One embodiment of the present invention includes so-called ‘lower’ alkyl chains of one to eight, preferably one to six carbon atoms. Thus, for example, C₁-C₆ alkyl represents a lower alkyl chain as hereinabove described.

As used herein the term “alkenyl” refers to a straight or branched chain aliphatic hydrocarbon having two to twelve carbon atoms, preferably two to eight carbon atoms, and containing one or more carbon-to-carbon double bonds, which may be optionally substituted as herein further described, with multiple degrees of substitution being allowed. Examples of “alkenyl” as used herein include, but are not limited to, vinyl, and allyl.

As used herein the term “alkynyl” refers to a straight or branched chain aliphatic hydrocarbon having two to twelve carbon atoms, preferably two to eight carbon atoms, and containing one or more carbon-to-carbon triple bonds, which may be optionally substituted as herein further described, with multiple degrees of substitution being allowed. An example of “alkynyl” as used herein includes, but is not limited to, ethynyl.

As used herein, the term “cycloalkyl” refers to a fully saturated optionally substituted three- to twelve-membered, preferably three- to eight-membered, monocyclic, bicyclic, Spiro, or bridged hydrocarbon ring, with multiple degrees of substitution being allowed. Exemplary “cycloalkyl” groups as used herein include, but are not limited to, cyclopropyl, cyclobutyl, cyclopentyl, cyclohexyl, and cycloheptyl.

Similarly, as used herein, the terms “cycloalkenyl” and “cycloalkynyl” refer to optionally substituted, partially saturated but non-aromatic, three-to-twelve membered, preferably either five- to eight-membered or seven- to ten-membered, monocyclic, bicyclic, Spiro, or bridged hydrocarbon rings, with one or more degrees of unsaturation, and with multiple degrees of substitution being allowed.

As used herein, the term “heterocycle” or “heterocyclyl” refers to an optionally substituted mono- or polycyclic ring system, optionally containing one or more degrees of unsaturation and also containing one or more heteroatoms, which may be optionally substituted as herein further described, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably three- to eight-membered and is either fully saturated or has one or more degrees of unsaturation. Such rings may be optionally fused or spiro with one or more of another heterocyclic ring(s) or cycloalkyl ring(s). Examples of “heterocyclic” groups as used herein include, but are not limited to, tetrahydrofuran, pyran, 1,4-dioxane, 1,3-dioxane, piperidine, pyrrolidine, morpholine, tetrahydrothiopyran, and tetrahydrothiophene.

As used herein, the term “aryl” refers to a univalent benzene ring or fused benzene ring system, which may be optionally substituted as herein further described, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and hexahydrocyclopenta-cyclooctene.

As used herein, the term “aralkyl” refers to an “aryl” group as herein defined attached through an alkylene linker.

As used herein, the term “heteroaryl” refers to a monocyclic five to seven membered aromatic ring, or to a fused bicyclic aromatic ring system comprising two of such aromatic rings, which may be optionally substituted as herein further described, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyridine, and pyrazolopyrimidine.

As used herein, the term “heteroaralkyl” refers to an “heteroaryl” group as herein defined attached through an alkylene linker.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein the term “alkoxy” refers to a group —OR^{a}, where R^{a} is alkyl as defined above.

As used herein the term “oxo” refers to a group ═O.

As used herein the term “nitro” refers to a group —NO₂.

As used herein the term “cyano” refers to a group —CN.

As used herein the term “azido” refers to a group —N₃.

As used herein “amino” refers to a group —NR^{a}R^{b}, where each of R^{a} and R^{b} individually is hydrogen, alkyl, alkenyl, alkynyl, cycloalkyl, aryl, heterocylcyl, or heteroaryl. As used herein, when either R^{a} or R^{b} is other than hydrogen, such a group may be referred to as a “substituted amino” or, for example if R^{a} is H and R^{b} is alkyl, as an “alkylamino.”

As used herein, the term “hydroxyl” refers to a group —OH.

One embodiment of the present invention includes a compound of Formula I:

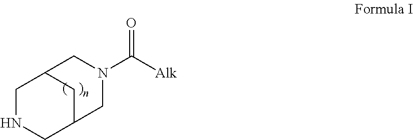

wherein n is 0 or 1;
Alk is alkyl, cycloalkyl, alkenyl, cycloalkenyl, alkynyl, or cycloalkynyl, each of which may be substituted with one, two, or three of alkyl, substituted alkyl, alkenyl, substituted alkenyl, alkynyl, substituted alkynyl, heterocyclyl, substituted heterocyclyl, cycloalkyl, substituted cycloalkyl, aryl, heteroaryl, substituted aryl, substituted heteroaryl, alkylaryl, alkylheteroaryl, substituted alkylaryl, substituted alkylheteroaryl, arylalkyl, heteroarylalkyl, substituted arylalkyl, substituted heteroarylalkyl, halogen, —OR′, ═O, —NR′R″, haloalkyl, —CN, —NO₂, —SR′, —N₃, —C(═O)NR′R″, —NR′C(═O)R″, —C(═O)R′, —C(═O)OR′, —OC(═O)R′, —OC(═O)NR′R″, —NR′C(═O)OR″, —SO₂R′, —SO₂NR′R″, and —NR′SO₂R″,
where R′ and R″ are independently selected from hydrogen, alkyl, cycloalkyl, heterocyclyl, aryl, heteroaryl, or arylalkyl,
or R′ and R″ and the atoms to which they are attached together can form a three- to eight-membered heterocyclic ring,
wherein the term “substituted”, as applied to alkyl, alkenyl, alkynyl, heterocyclyl, cycloalkyl, aryl, heteroaryl, alkylaryl, alkylheteroaryl, arylalkyl, and heteroarylalkyl, refers to substitution with one or more alkyl, aryl, heteroaryl, halogen, —OR′, or —NR′R″ groups, where R′ and R″ are as defined;
or a pharmaceutically acceptable salt thereof.

One embodiment of the present invention includes wherein:

n has the value of 0 or 1; and
Alk is methyl, ethyl, n-propyl, isopropyl, 1-propenyl, allyl, n-butyl, 1-butenyl, 2-butenyl, 3-butenyl, isobutyl, sec-butyl, tert-butyl, cyclopropyl, cyclobutyl, cyclopentyl, cyclopentenyl, cyclohexyl, cyclohexenyl, spirobicyclohexyl, cycloheptyl, bicycloheptyl, bicycloheptenyl, cyclooctyl, bicyclooctyl, or bicyclooctenyl, each of which may be substituted with one, two, or three of alkyl, aryl, heteroaryl, substituted aryl, substituted heteroaryl, halogen, —OR′, ═O, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —OC(═O)NR′R″, —NR′C(═O)OR″, —SO₂R′, —SO₂NR′R″, or —NR′SO₂R″,
where R′ and R″ are defined as in claim 1, wherein the term “substituted” is defined as in claim 1, or a pharmaceutically acceptable salt thereof.

One embodiment of the present invention includes pharmaceutically acceptable salts, wherein Alk is methyl, ethyl or n-propyl.

Thus, one embodiment of the present invention includes a pharmaceutically acceptable salt of Formula Ia:

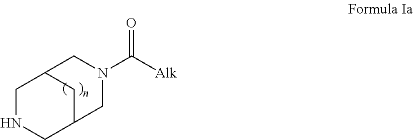

wherein n is 0 or 1; and
Alk is methyl, ethyl, or n-propyl.

One embodiment of the present invention includes pharmaceutically acceptable salts, wherein Alk is cycloalkyl, in a further embodiment, cyclopropyl.

Thus, one embodiment of the present invention includes a pharmaceutically acceptable salt of Formula Ia:

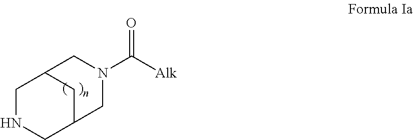

wherein n is 0 or 1; and
Alk is cycloalkyl. In a further embodiment, Alk is cyclopropyl. In still a further embodiment, Alk is a cyclopropyl substituted with one or more halogen.

One embodiment of the present invention includes compounds wherein n is 0.

One embodiment of the present invention includes compounds wherein n is 1.

One embodiment of the present invention includes a compound selected from the group consisting of:
N-(acetyl)-3,7-diazabicyclo[3.3.0]octane,
N-(fluoroacetyl)-3,7-diazabicyclo[3.3.0]octane,
N-(methoxyacetyl)-3,7-diazabicyclo[3.3.0]octane,
N-(2-phenyl-2-methoxyacetyl)-3,7-diazabicyclo[3.3.0]octane,
N-(hydroxyacetyl)-3,7-diazabicyclo[3.3.0]octane,
N-(difluoroacetyl)-3,7-diazabicyclo[3.3.0]octane,
N-(carbamoylacetyl)-3,7-diazabicyclo[3.3.0]octane,
N-(methylsulfonylacetyl)-3,7-diazabicyclo[3.3.0]octane,
N-(phenylsulfonylacetyl)-3,7-diazabicyclo[3.3.0]octane,
N-(cyclopropylacetyl)-3,7-diazabicyclo[3.3.0]octane,
N-(propanoyl)-3,7-diazabicyclo[3.3.0]octane,
N-(3-fluoropropanoyl)-3,7-diazabicyclo[3.3.0]octane,
N-(3-methoxpropanoyl)-3,7-diazabicyclo[3.3.0]octane,
N-(2,2-difluoropropanoyl)-3,7-diazabicyclo[3.3.0]octane,
N-(2-propenoyl)-3,7-diazabicyclo[3.3.0]octane,
N-(butanoyl)-3,7-diazabicyclo[3.3.0]octane,
N-(2-butenoyl)-3,7-diazabicyclo[3.3.0]octane,
N-(3-butenoyl)-3,7-diazabicyclo[3.3.0]octane,
N-(2-methylpropanoyl)-3,7-diazabicyclo[3.3.0]octane,
N-(2-fluoro-2-methylpropanoyl)-3,7-diazabicyclo[3.3.0]octane,
N-(pentanoyl)-3,7-diazabicyclo[3.3.0]octane,
N-(3-methylbutanoyl)-3,7-diazabicyclo[3.3.0]octane,
N-(2-methylbutanoyl)-3,7-diazabicyclo[3.3.0]octane,
N-(2,2-dimethylpropanoyl)-3,7-diazabicyclo[3.3.0]octane,
N-(3-methyl-2-butenoyl)-3,7-diazabicyclo[3.3.0]octane,
N-(3-pentenoyl)-3,7-diazabicyclo[3.3.0]octane,
N-(cyclopropylcarbonyl)-3,7-diazabicyclo[3.3.0]octane,
N-(2-fluorocyclopropylcarbonyl)-3,7-diazabicyclo[3.3.0]octane,
N-(1-methylcyclopropylcarbonyl)-3,7-diazabicyclo[3.3.0]octane,
N-(1-hydroxycyclopropylcarbonyl)-3,7-diazabicyclo[3.3.0]octane,
N-(1-cyanocyclopropylcarbonyl)-3,7-diazabicyclo[3.3.0]octane,
N-(2-methylcyclopropylcarbonyl)-3,7-diazabicyclo[3.3.0]octane,
N-(2,2-difluorocyclopropylcarbonyl)-3,7-diazabicyclo[3.3.0]octane,
N-(2,2-dimethylcyclopropylcarbonyl)-3,7-diazabicyclo[3.3.0]octane,
N-(2,2,3,3-tetramethylcyclopropylcarbonyl)-3,7-diazabicyclo[3.3.0]octane,
N-(cyclobutylcarbonyl)-3,7-diazabicyclo[3.3.0]octane,
N-(3-fluorocyclobutylcarbonyl)-3,7-diazabicyclo[3.3.0]octane,
N-(3,3-difluorocyclobutylcarbonyl)-3,7-diazabicyclo[3.3.0]octane,
N-(3,3-dimethylcyclobutylcarbonyl)-3,7-diazabicyclo[3.3.0]octane,
N-(3-methoxycyclobutylcarbonyl)-3,7-diazabicyclo[3.3.0]octane,
N-(cyclopentylcarbonyl)-3,7-diazabicyclo[3.3.0]octane,
N-(1-cyclopentenylcarbonyl)-3,7-diazabicyclo[3.3.0]octane,
N-(2-cyclopentenylcarbonyl)-3,7-diazabicyclo[3.3.0]octane,
N-(3-cyclopentenylcarbonyl)-3,7-diazabicyclo[3.3.0]octane,
N-(cyclohexylcarbonyl)-3,7-diazabicyclo[3.3.0]octane,
N-(3-cyclohexenylcarbonyl)-3,7-diazabicyclo[3.3.0]octane,
N-(norbornylcarbonyl)-3,7-diazabicyclo[3.3.0]octane,
N-(spiro[2.3]hexyl-1-carbonyl)-3,7-diazabicyclo[3.3.0]octane,
N-(bicyclo[4.1.0]heptyl-7-carbonyl)-3,7-diazabicyclo[3.3.0]octane,
N-(bicyclo[2.2.1]hept-5-enyl-2-carbonyl)-3,7-diazabicyclo[3.3.0]octane, and
N-(bicyclo[2.2.2]oct-5-enyl-2-carbonyl)-3,7-diazabicyclo[3.3.0]octane,
   or a pharmaceutically acceptable salt thereof.

One embodiment of the present invention includes a compound selected from the group consisting of:
N-(acetyl)-3,7-diazabicyclo[3.3.1]nonane,
N-(fluoroacetyl)-3,7-diazabicyclo[3.3.1]nonane,
N-(methoxyacetyl)-3,7-diazabicyclo[3.3.1]nonane,
N-(2-phenyl-2-methoxyacetyl)-3,7-diazabicyclo[3.3.1]nonane,
N-(hydroxyacetyl)-3,7-diazabicyclo[3.3.1]nonane,
N-(difluoroacetyl)-3,7-diazabicyclo[3.3.1]nonane,
N-(carbamoylacetyl)-3,7-diazabicyclo[3.3.1]nonane,
N-(methylsulfonylacetyl)-3,7-diazabicyclo[3.3.1]nonane,
N-(phenylsulfonylacetyl)-3,7-diazabicyclo[3.3.1]nonane,
N-(cyclopropylacetyl)-3,7-diazabicyclo[3.3.1]nonane,
N-(propanoyl)-3,7-diazabicyclo[3.3.1]nonane,
N-(3-fluoropropanoyl)-3,7-diazabicyclo[3.3.1]nonane,
N-(3-methoxypropanoyl)-3,7-diazabicyclo[3.3.1]nonane,
N-(2,2-difluoropropanoyl)-3,7-diazabicyclo[3.3.1]nonane,
N-(2-propenoyl)-3,7-diazabicyclo[3.3.1]nonane,
N-(butanoyl)-3,7-diazabicyclo[3.3.1]nonane,
N-(2-butenoyl)-3,7-diazabicyclo[3.3.1]nonane,
N-(3-butenoyl)-3,7-diazabicyclo[3.3.1]nonane,
N-(2-methylpropanoyl)-3,7-diazabicyclo[3.3.1]nonane,
N-(2-fluoro-2-methylpropanoyl)-3,7-diazabicyclo[3.3.1]nonane,
N-(pentanoyl)-3,7-diazabicyclo[3.3.1]nonane,
N-(3-methylbutanoyl)-3,7-diazabicyclo[3.3.1]nonane,
N-(2-methylbutanoyl)-3,7-diazabicyclo[3.3.1]nonane,
N-(2,2-dimethylpropanoyl)-3,7-diazabicyclo[3.3.1]nonane,
N-(3-methyl-2-butenoyl)-3,7-diazabicyclo[3.3.1]nonane,
N-(3-pentenoyl)-3,7-diazabicyclo[3.3.1]nonane,
N-(cyclopropylcarbonyl)-3,7-diazabicyclo[3.3.1]nonane,
N-(2-fluorocyclopropylcarbonyl)-3,7-diazabicyclo[3.3.1]nonane,
N-(1-methylcyclopropylcarbonyl)-3,7-diazabicyclo[3.3.1]nonane,
N-(1-hydroxycyclopropylcarbonyl)-3,7-diazabicyclo[3.3.1]nonane,
N-(1-cyanocyclopropylcarbonyl)-3,7-diazabicyclo[3.3.1]nonane,
N-(2-methylcyclopropylcarbonyl)-3,7-diazabicyclo[3.3.1]nonane,
N-(2,2-difluorocyclopropylcarbonyl)-3,7-diazabicyclo[3.3.1]nonane,
N-(2,2-dimethylcyclopropylcarbonyl)-3,7-diazabicyclo[3.3.1]nonane,
N-(2,2,3,3-tetramethylcyclopropylcarbonyl)-3,7-diazabicyclo[3.3.1]nonane,
N-(cyclobutylcarbonyl)-3,7-diazabicyclo[3.3.1]nonane,
N-(3-fluorocyclobutylcarbonyl)-3,7-diazabicyclo[3.3.1]nonane,
N-(3,3-difluorocyclobutylcarbonyl)-3,7-diazabicyclo[3.3.1]nonane,
N-(3,3-dimethylcyclobutylcarbonyl)-3,7-diazabicyclo[3.3.1]nonane,
N-(3-methoxycyclobutylcarbonyl)-3,7-diazabicyclo[3.3.1]nonane,
N-(cyclopentylcarbonyl)-3,7-diazabicyclo[3.3.1]nonane,
N-(1-cyclopentenylcarbonyl)-3,7-diazabicyclo[3.3.1]nonane,
N-(2-cyclopentenylcarbonyl)-3,7-diazabicyclo[3.3.1]nonane,
N-(3-cyclopentenylcarbonyl)-3,7-diazabicyclo[3.3.1]nonane,
N-(cyclohexylcarbonyl)-3,7-diazabicyclo[3.3.1]nonane,
N-(3-cyclohexenylcarbonyl)-3,7-diazabicyclo[3.3.1]nonane,
N-(norbornylcarbonyl)-3,7-diazabicyclo[3.3.1]nonane,
N-(spiro[2.3]hexyl-1-carbonyl)-3,7-diazabicyclo[3.3.1]nonane,
N-(bicyclo[4.1.0]heptyl-7-carbonyl)-3,7-diazabicyclo[3.3.1]nonane,
N-(bicyclo[2.2.1]hept-5-enyl-2-carbonyl)-3,7-diazabicyclo[3.3.1]nonane, and
N-(bicyclo[2.2.2]oct-5-enyl-2-carbonyl)-3,7-diazabicyclo[3.3.1]nonane,
   or a pharmaceutically acceptable salt thereof.

One embodiment of the present invention includes use of a compound of the present invention in the manufacture of a medicament.

One embodiment of the present invention includes a method for the treatment or prevention of central nervous system disorders and dysfunctions, comprising administering to a mammal in need of such treatment, a therapeutically effective amount of the compound of the present invention. More specifically, the disorder or dysfunction may be selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, schizophreniform disorder, schizoaffective disorder, cognitive deficits in schizophrenia, and cognitive dysfunction in schizophrenia. Still further, the disorder may be selected from the group consisting of mild to moderate dementia of the Alzheimer's type, attention deficit disorder, attention deficit hyperactivity disorder, mild cognitive impairment, age-associated memory impairment, cognitive deficits in schizophrenia, and cognitive dysfunction in schizophrenia.

One embodiment of the present invention includes a pharmaceutical composition comprising a therapeutically effective amount of a compound of the present invention and one or more pharmaceutically acceptable carriers.

One embodiment of the present invention includes the use of a pharmaceutical composition of the present invention in the manufacture of a medicament for treatment of central nervous system disorders and dysfunctions.

Another embodiment of the present invention includes a compound as herein described with reference to any one of the Examples.

Another embodiment of the present invention includes a compound of the present invention for use as an active therapeutic substance.

Another embodiment of the present invention includes a compound of the present invention for use to modulate an NNR in a subject in need thereof.

Another embodiment of the present invention includes a compound of the present invention for use in the treatment or prevention of conditions or disorders mediated by NNR.

Another embodiment of the present invention includes a use of a compound of the present invention in the manufacture of a medicament for use of modulating NNR in a subject in need thereof.

Another embodiment of the present invention includes a use of a compound of the present invention in the manufacture of a medicament for use in the treatment or prevention of conditions or disorders mediated by NNR.

Another embodiment of the present invention includes a method of modulating NNR in a subject in need thereof through the administration of a compound of the present invention.

The scope of the present invention includes combinations of embodiments.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates. Representative salts are provided as described in U.S. Pat. Nos. 5,597,919 to Dull et al., 5,616,716 to Dull et al. and 5,663,356 to Ruecroft et al, each of which is herein incorporated by reference with regard to such salts.

As noted herein, the present invention includes specific representative compounds, which are identified herein with particularity.

One embodiment relates to N-(propanoyl)-3,7-diazabicyclo[3.3.0]octane, or a pharmaceutically acceptable salt thereof.

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of the present invention.

Those skilled in the art will recognize if a stereocenter exists. As noted hereinabove, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994).

The present invention also provides a method for the synthesis of compounds useful as intermediates in the preparation of compounds of the present invention along with methods for their preparation.

The compounds can be prepared according to the following methods using readily available starling materials and reagents. In these reactions, variants may be employed which are themselves known to those of ordinary skill in this art, but are not mentioned in greater detail.

### Description of General Synthetic Methods

The compounds of the present invention can be prepared via the coupling of mono-protected diazabicycle, namely one in which one of the two amine functional groups is rendered un-reactive by suitable derivatization, with a suitably functionalized aliphatic acid chloride or other reactive carboxylic acid derivative.

There are numerous methods for preparing the mono-protected diazabicycles used to prepare the compounds of the present invention. Methods for the synthesis of a suitably protected 3,7-diazabicyclo[3.3.0]octane are described in PCT WO 02/070523 to Colon-Cruz et al. and in U.S. application 2006/0019985 to Zhenkun et al., each of which is incorporated by reference with regard to such synthetic teaching, in which N-benzylmaleimide is condensed with either paraformaldehyde and N-benzylglycine or N-(methoxymethyl)-N-(trimethylsilylmethyl)benzylamine to produce 3,7-dibenzyl-3,7-diazabicyclo[3.3.0]octane-2,4-dione (also known as 2,5-dibenzyltetrahydropyrrolo[3,4-c]pyrrole-1,3-dione). Subsequent transformation of this intermediate can follow several paths. In one instance, treatment with α-chloroethylchloroformate produces 3-benzyl-3,7-diazabicyclo[3.3.0]octane-2,4-dione (also known as 2-benzyltetrahydropyrrolo[3,4-c]pyrrole-1,3-dione), which is then sequentially reduced (using borane-dimethylsulfide complex), converted into its N-(tert-butoxycarbonyl) derivative, and hydrogenated (to remove the second benzyl group). This produces N-(tert-butoxycarbonyl)-3,7-diazabicyclo[3.3.0]octane, which can be used in coupling with carboxylic acids, and their derivatives, to produce compounds of the present invention. Alternately, 3,7-dibenzyl-3,7-diazabicyclo[3.3.0]octane-2,4-dione can be reduced, such as with lithium aluminum hydride, partially hydrogenated, namely to remove one benzyl group, converted into its N-(tert-butoxycarbonyl) derivative, and hydrogenated, namely to remove the second benzyl group, thereby to produce N-(tert-butoxycarbonyl)-3,7-diazabicyclo[3.3.0]octane.

Other methods for installation and removal of the benzyl, tert-butoxycarbonyl, and other amine protecting groups are well known by those skilled in the art and are described further in T. W. Greene and P. G. M. Wuts, Protective Groups in Organic Synthesis, 3^{rd} Edition, John Wiley & Sons, New York (1999).

An alternative preparation of N-(tert-butoxycarbonyl)-3,7-diazabicyclo[3.3.0]octane has been described in U.S. applications 2004/0186107 to Schrimpf et al. and 2005/0101602 to Basha et al., each herein incorporated by reference with regard to such synthetic teaching, and involves the condensation of maleimide and N-(methoxymethyl)-N-(trimethylsilylmethyl)benzylamine to give 7-benzyl-3,7-diazabicyclo[3.3.0]octane-2,4-dione (also known as 5-benzyltetrahydropyrrolo[3,4-c]pyrrole-1,3-dione). Subsequent treatment with a reducing agent, such as lithium aluminum hydride, produces the 3-benzyl-3,7-diazabicyclo[3.3.0]octane, the free amine of which can be protected by a tert-butoxycabonyl group, followed by removal of the benzyl protecting group by hydrogenolysis.

Maleate esters can be used as alternatives to maleimides in these condensation reactions. Thus, according to PCT WO 96/007656 to Schaus et al., herein incorporated by reference with regard to such synthetic teaching, condensation of N-benzylglycine with paraformaldehyde and dimethyl maleate will give N-benzyl-cis-3,4-pyrrolidinedicarboxylic acid dimethyl ester. This compound can then be reduced, for example, with lithium aluminum hydride, to give the diol, which can be further reacted with methanesulfonyl chloride in the presence of triethylamine to produce the corresponding dimesylate. Further treatment with ammonia and heat provides the N-benzyl protected 3,7-diazabicyclo[3.3.0]octane. As described above, this can be converted into N-(tert-butoxycarbonyl)-3,7-diazabicyclo[3.3.0]octane.

Suitable derivatives of 3,7-diazabicyclo[3.3.1]nonane (bispidine) can be used to make compounds of the present invention. One such derivative is N-(tert-butoxycarbonyl)-3,7-diazabicyclo[3.3.1]nonane, which can be made in a variety of ways. One synthesis proceeds through N-benzyl-N′-(tert-butoxycarbonyl)-3,7-diazabicyclo[3.3.1]nonane, described by Stead et al. in Org. Lett. 7: 4459 (2005), herein incorporated by reference with regard to such teaching. Thus a Mannich reaction between N-(tert-butoxycarbonyl)piperidin-4-one, benzylamine and paraformaldehyde affords N-benzyl-N′-(tert-butoxycarbonyl)-3,7-diazabicyclo[3.3.1]nonan-9-one, which can be treated sequentially with p-toluenesulfonhydrazide and sodium borohydride, namely to remove the carbonyl oxygen, to give N-benzyl-N′-(tert-butoxycarbonyl)-3,7-diazabicyclo[3.3.1]nonane. The benzyl group can be removed by hydrogenolysis to provide N-(tert-butoxycarbonyl)-3,7-diazabicyclo[3.3.1]nonane. Alternative syntheses of diazabicyclo[3.3.1]nonanes, suitable for conversion to either N-(tert-butoxycarbonyl)-3,7-diazabicyclo[3.3.1]nonane or another mono-protected derivative, have been described by Jeyaraman and Avila in Chem. Rev. 81(2): 149-174 (1981) and in U.S. Pat. No. 5,468,858 to Berlin et al, each of which is herein incorporated by reference with regard to such synthesis.

One means of making amides of the present invention is to couple the either N-(tert-butoxycarbonyl)-3,7-diazabicyclo[3.3.0]octane or N-(tert-butoxycarbonyl)-3,7-diazabicyclo[3.3.1]nonane with a suitably functionalized carboxylic acid and then remove the tert-butoxycarbonyl protecting group. Many such carboxylic acids are commercially available, and others can be easily prepared by procedures known to those skilled in the art. The condensation of an amine and a carboxylic acid, to produce an amide, typically requires the use of a suitable activating agent, such as N,N′-dicyclohexylcarbodiimide (DCC), (benzotriazol-1-yloxy)tris(dimethylamino)phosphonium hexafluorophosphate (BOP), (benzotriazol-1-yloxy)tripyrrolidinophosphonium hexafluorophosphate (PyBOP), O-(benzotriazol-1-yl)-N,N,N′,N′-bis(tetramethylene)uronium hexafluorophosphate (HBPyU), O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), or (1-ethyl-3-(3-dimethylaminopropyl)carbodiimide) (EDCI) with 1-hydroxybenzotriazole (HOBt). Other activating agents are well known to those skilled in the art, for example, see Kiso and Yajima, Peptides, pp 39-91, Academic Press, San Diego, Calif. (1995), herein incorporated by reference with regard to such agents.

Alternatively, the amide bond can be formed by coupling a mono-protected diazabicycle with a suitably functionalized acid chloride, which may be available commercially or may be prepared by conversion of the suitably functionalized carboxylic acid. The acid chloride may be prepared by treatment of the appropriate carboxylic acid with, among other reagents, thionyl chloride or oxalyl chloride.

As will be appreciated by those skilled in the art, the use of certain carboxylic acids containing ancillary reactive functional groups may require additional protection/deprotection steps to prevent interference with the amide bond formation. Such protection/deprotection steps are well known in the art (for example, see T. W. Green and P. G. M. Wuts (1991) Protecting Groups in Organic Synthesis, John Wiley & Sons).

After amide formation, removal of the protecting group, for example, the tert-butoxycarbonyl group, with acid, either aqueous or anhydrous, will afford the compounds of the present invention.

Those skilled in the art of organic synthesis will appreciate that there exist multiple means of producing compounds of the present invention which are labeled with a radioisotope appropriate to various diagnostic uses. Thus, condensation of a ¹¹C- or ¹⁸F-labeled aliphatic carboxylic acid with either N-(tert-butoxycarbonyl)-3,7-diazabicyclo[3.3.0]octane or N-(tert-butoxycarbonyl)-3,7-diazabicyclo[3.3.1]nonane, using the methods described above, and subsequent removal of the tert-butoxycarbonyl group will produce a compound suitable for use in positron emission tomography.

As will be appreciated by those skilled in the art throughout the present specification, the number and nature of substituents on rings in the compounds of the present invention will be selected so as to avoid sterically undesirable combinations.

Certain compound names of the present invention were generated with the aid of computer software (ACDLabs 8.0/Name (IUPAC)).

### Methods of Treatment

The compounds of the present invention can be used for the prevention or treatment of various conditions or disorders for which other types of nicotinic compounds have been proposed or are shown to be useful as therapeutics, such as CNS disorders, inflammation, inflammatory response associated with bacterial and/or viral infection, pain, metabolic syndrome, autoimmune disorders or other disorders described in further detail herein. The compounds can also be used as a diagnostic agent in receptor binding studies (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther 279: 1413 (1996), Lippiello et al., J. Pharmacol. Exp. Ther 279: 1422 (1996), Damaj et al., J. Pharmacol. Exp. Ther 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. Nos. 5,583,140 to Bencherif et al., 5,597,919 to Dull et al., 5,604,231 to Smith et al. and 5,852,041 to Cosford et al.

### CNS Disorders

A compound of the present invention or a pharmaceutically acceptable salt thereof, or a pharmaceutical composition comprising said compounds are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, oppositional defiant disorder, Tourette's syndrome, autism, drug and alcohol addiction, tobacco addiction, obesity, cachexia, psoriasis, lupus, acute cholangitis, aphthous stomatitis, ulcers, asthma, ulcerative colitis, inflammatory bowel disease, Crohn's disease, spastic dystonia, diarrhea, constipation, pouchitis, viral pneumonitis, arthritis, including, rheumatoid arthritis and osteoarthritis, endotoxaemia, sepsis, atherosclerosis, idiopathic pulmonary fibrosis, acute pain, chronic pain, neuropathies, urinary incontinence, diabetes and neoplasias.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

One embodiment relates to treating CNS disorders in a subject in need thereof comprising administering to said subject a compound of the present invention.

In another embodiment, the CNS disorders are selected from cognitive dysfunction in schizophrenia (CDS), Alzheimer's Disease (AD), attention deficit disorder (ADD), pre-senile dementia (also known as early onset of Alzheimer's Disease), dementia of the Alzheimer's type, mild cognitive impairment, age associated memory impairment and attention deficit hyperactivity disorder (ADHD).

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The inflammatory reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, Crohn's disease, ulcerative colitis, acute cholangitis, aphteous stomatitis, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties.

The compounds of the present invention may also be used as adjunct therapy in combination with existing therapies to manage bacterial, viral and fungal infections, such as antibiotics, antivirals and antifungals. Antitoxins may also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine and gout). Inflammatory pain may be of diverse genesis, including arthritis and rheumatoid disease, teno-synovitis and vasculitis. Neuropathic pain includes trigeminal or herpetic neuralgia, diabetic neuropathy pain, causalgia, low back pain and deafferentation syndromes such as brachial plexus avulsion.

One embodiment relates to treating pain in a subject in need thereof comprising administering to said subject a compound of the present invention.

### Other Disorders

In addition to treating CNS disorders, inflammation, and pain, the compounds of the present invention may be also used to prevent or treat certain other conditions, diseases, and disorders in which NNRs play a role. Examples include autoimmune disorders such as Lupus, disorders associated with cytokine release, cachexia secondary to infection (e.g., as occurs in AIDS, AIDS related complex and neoplasia), obesity, pemphitis, urinary incontinence, retinal diseases, infectious diseases, myasthenia, Eaton-Lambert syndrome, hypertension, osteoporosis, vasoconstriction, vasodilatation, cardiac arrhythmias, type I diabetes, bulimia, anorexia as well as those indications set forth in published PCT application WO 98/25619. The compounds of this invention may also be administered to treat convulsions such as those that are symptomatic of epilepsy, and to treat conditions such as syphillis and Creutzfeld-Jakob disease.

### Diagnostic Uses

The compounds may be used in diagnostic compositions, such as probes, particularly when they are modified to include appropriate labels. The probes may be used, for example, to determine the relative number and/or function of specific receptors, particularly the α4β2 receptor subtype. For this purpose the compounds of the present invention most preferably are labeled with a radioactive isotopic moiety such as ¹¹C, ¹⁸F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 min for ¹¹C, about 109 min for ¹⁸F, about 13 h for ¹²³I, and about 16 h for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds may be administered using known techniques. See, for example, U.S. Pat. No. 5,969,144 to London et al. The compounds may be administered in compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality of selected NNR subtypes. In addition to humans, the compounds may also be administered to animals, such as mice, rats, horses, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al.

The radiolabeled compounds bind with high affinity to selective NNR subtypes (e.g., α4β2) and preferably exhibit negligible non-specific binding to other nicotinic cholinergic receptor subtypes (e.g., those receptor subtypes associated with muscle and ganglia). As such, the compounds can be used as agents for noninvasive imaging of nicotinic cholinergic receptor subtypes within the body of a subject, particularly within the brain for diagnosis associated with a variety of CNS diseases and disorders.

In one aspect, the diagnostic compositions may be used in a method to diagnose disease in a subject, such as a human patient. The method involves administering to that patient a detectably labeled compound as described herein, and detecting the binding of that compound to selected NNR subtypes (e.g., α4β2 receptor subtypes). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that may be treated include those that are set forth in U.S. Pat. No. 5,952,339 to Bencherif et al.

In another aspect, the diagnostic compositions can be used in a method to monitor selective nicotinic receptor subtypes of a subject, such as a human patient. The method involves administering a detectably labeled compound as described herein to that patient and detecting the binding of that compound to selected nicotinic receptor subtypes namely, the α4β2 receptor subtypes.

### Receptor Binding

The compounds of this invention may be used as reference ligands in binding assays for compounds which bind to NNR subtypes, particularly the α4β2 receptor subtypes. For this purpose the compounds of this invention are preferably labeled with a radioactive isotopic moiety such as ³H, or ¹⁴C. Examples of such binding assays are described in detail below.

### Pharmaceutical Compositions

Although it is possible to administer the compound of the present invention in the form of a bulk active chemical, it is preferred to administer the compound in the form of a pharmaceutical composition or formulation. Thus, in one aspect the present invention relates to pharmaceutical compositions comprising the compound of the present invention and one or more pharmaceutically acceptable carrier, diluent, or excipient. Another aspect of the invention provides a process for the preparation of a pharmaceutical composition including admixing the compound of the present invention with one or more pharmaceutically acceptable carrier, diluent, or excipient.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions may also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline.

The compositions may also be administered using other means, for example, rectal administration. Compositions useful for rectal administration, such as suppositories, are well known to those of skill in the art. The compounds may also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses forms.

The administration of the pharmaceutical compositions described herein can be intermittent, or at a gradual, continuous, constant or controlled rate. The pharmaceutical compositions may be administered to a warm-blooded animal, for example, a mammal such as a mouse, rat, cat, rabbit, horses, dog, pig, cow, or monkey; but advantageously is administered to a human being. The compounds of the present invention may be used in the treatment of a variety of disorders and conditions and, as such, may be used in combination with a variety of other therapeutic agents useful in the treatment or prophylaxis of those disorders. Thus, one embodiment of the present invention relates to the administration of a compound of the present invention in combination with other therapeutic agents. For example, a compound of the present invention may be used in combination with other NNR ligands (such as varenidine), antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of therapeutic agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other therapeutic agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention relates to combination therapy comprising administering to the subject a therapeutically or prophylactically effective amount of a compound of the present invention and one or more other therapeutic agents including chemotherapeutics, radiation therapeutic agents, gene therapeutic agents, or agents used in immunotherapy.

As used herein, the terms “prevention” or “prophylaxis” include any degree of reducing the progression of or delaying the onset of a disease, disorder, or condition. The term includes providing protective effects against a particular disease, disorder, or condition as well as amelioration or reduction of the recurrence of the disease, disorder, or condition. Thus, in another aspect, the invention provides a method for treating a subject having or at risk of developing or experiencing a recurrence of a NNR or nAChR mediated disorder. The compounds and pharmaceutical compositions of the invention may be used to achieve a beneficial therapeutic or prophylactic effect, for example, in a subject with a CNS dysfunction.

### Biological Assays

### Example 1

### Radioligand Binding at CNS nAChRs

α4β2 nAChR Subtype

Preparation of Membranes from Rat Cortex: Rats (Female, Sprague-Dawley), weighing 150-250 g, were maintained on a 12 h light/dark cycle and were allowed free access to water and food supplied by PMI Nutrition International, Inc. Animals were anesthetized with 70% CO₂, and then decapitated. Brains were removed and placed on an ice-cold platform. The cerebral cortex was removed and placed in 20 volumes (weight:volume) of ice-cold preparative buffer (137 mM NaCl, 10.7 mM KCl, 5.8 mM KH₂PO₄, 8 mM Na₂HPO₄, 20 mM HEPES (free acid), 5 mM iodoacetamide, 1.6 mM EDTA, pH 7.4); PMSF, dissolved in methanol to a final concentration of 100 μM, was added and the suspension was homogenized by Polytron. The homogenate was centrifuged at 18,000×g for 20 min at 4° C. and the resulting pellet was re-suspended in 20 volumes of ice-cold water. After 60 min incubation on ice, a new pellet was collected by centrifugation at 18,000×g for 20 min at 4° C. The final pellet was re-suspended in 10 volumes of buffer and stored at −20° C.

Preparation of membranes from SH-EP1/human α4β2 clonal cells: Cell pellets from 40 150 mm culture dishes were pooled, and homogenized by Polytron (Kinematica GmbH, Switzerland) in 20 milliliters of ice-cold preparative buffer. The homogenate was centrifuged at 48,000 g for 20 minutes at 4° C. The resulting pellet was re-suspended in 20 mL of ice-cold preparative buffer and stored at −20° C.

On the day of the assay, the frozen membranes were thawed and spun at 48,000×g for 20 min. The supernatant was decanted and discarded. The pellet was resuspended in Dulbecco's phosphate buffered saline (PBS, Life Technologies) pH 7.4 and homogenized with the Polytron for 6 seconds. Protein concentrations were determined using a Pierce BCA Protein Assay Kit, with bovine serum albumin as the standard (Pierce Chemical Company, Rockford, Ill.).

Membrane preparations (approximately 50 μg for human and 200-300 μg protein for rat α4β2) were incubated in PBS (50 μL and 100 μL respectively) in the presence of competitor compound (0.01 nM to 100 μM) and 5 nM [³H]nicotine for 2-3 hours on ice. Incubation was terminated by rapid filtration on a multi-manifold tissue harvester (Brandel, Gaithersburg, Md.) using GF/B filters presoaked in 0.33% polyethyleneimine (w/v) to reduce non-specific binding. Tissue was rinsed 3 times in PBS, pH 7.4. Scintillation fluid was added to filters containing the washed tissue and allowed to equilibrate. Filters were then counted to determine radioactivity bound to the membranes by liquid scintillation counting (2200CA Tri-Carb LSC, Packard Instruments, 50% efficiency or Wallac Trilux 1450 MicroBeta, 40% efficiency, Perkin Elmer).

Data were expressed as disintegrations per minute (DPMs). Within each assay, each point had 2-3 replicates. The replicates for each point were averaged and plotted against the log of the drug concentration. IC₅₀, which is the concentration of the compound that produces 50% inhibition of binding, was determined by least squares non-linear regression. Ki values were calculated using the Cheng-Prussof equation (1973):

Ki=IC₅₀/(1+N/Kd)

where N is the concentration of [³H]nicotine and Kd is the affinity of nicotine (3 nM, determined in a separate experiment).

α7 nAChR Subtype

Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, then decapitated. Brains were removed and placed on an ice-cold platform. The hippocampus was removed and placed in 10 volumes (weight:volume) of ice-cold preparative buffer (137 mM NaCl, 10.7 mM KCl, 5.8 mM KH₂PO₄, 8 mM Na₂HPO₄, 20 mM HEPES (free acid), 5 mM iodoacetamide, 1.6 mM EDTA, pH 7.4); PMSF, dissolved in methanol to a final concentration of 100 μM, was added and the tissue suspension was homogenized by Polytron. The homogenate was centrifuged at 18,000×g for 20 min at 4° C. and the resulting pellet was re-suspended in 10 volumes of ice-cold water. After 60 min incubation on ice, a new pellet was collected by centrifugation at 18,000×g for 20 min at 4° C. The final pellet was re-suspended in 10 volumes of buffer and stored at −20° C. On the day of the assay, tissue was thawed, centrifuged at 18,000×g for 20 min, and then re-suspended in ice-cold PBS (Dulbecco's Phosphate Buffered Saline, 138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH 7.4) to a final concentration of approximately 2 mg protein/mL. Protein was determined by the method of Lowry et al., J. Biol. Chem. 193: 265 (1951), using bovine serum albumin as the standard.

The binding of [³H]MLA was measured using a modification of the methods of Davies et al., Neuropharmacol. 38: 679 (1999). [³H]MLA (Specific Activity=25-35 Ci/mmol) was obtained from Tocris. The binding of [³H]MLA was determined using a 2 h incubation at 21° C. Incubations were conducted in 48-well micro-titre plates and contained about 200 μg of protein per well in a final incubation volume of 300 μL. The incubation buffer was PBS and the final concentration of [³H]MLA was 5 nM. The binding reaction was terminated by filtration of the protein containing bound ligand onto glass fiber filters (GF/B, Brandel) using a Brandel Tissue Harvester at room temperature. Filters were soaked in de-ionized water containing 0.33% polyethyleneimine to reduce non-specific binding. Each filter was washed with PBS (3×1 mL) at room temperature. Non-specific binding was determined by inclusion of 50 μM non-radioactive MLA in selected wells.

The inhibition of [³H]MLA binding by test compounds was determined by including seven different concentrations of the test compound in selected wells. Each concentration was replicated in triplicate. IC₅₀ values were estimated as the concentration of compound that inhibited 50 percent of specific [³H]MLA binding. Inhibition constants (Ki values), reported in nM, were calculated from the IC₅₀ values using the method of Cheng et al., Biochem. Pharmacol. 22: 3099-3108 (1973).

### Example 2

### Determination of Dopamine Release

Dopamine release was measured using striatal synaptosomes obtained from rat brain, according to the procedures set forth by Rapier et al., J. Neurochem. 54: 937 (1990). Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, then decapitated. The brains were quickly removed and the striata dissected. Striatal tissue from each of 2 rats was pooled and homogenized in ice-cold 0.32 M sucrose (5 mL) containing 5 mM HEPES, pH 7.4, using a glass/glass homogenizer. The tissue was then centrifuged at 1,000×g for 10 min. The pellet was discarded and the supernatant was centrifuged at 12,000×g for 20 min. The resulting pellet was re-suspended in perfusion buffer containing monoamine oxidase inhibitors (128 mM NaCl, 1.2 mM KH₂PO₄, 2.4 mM KCl, 3.2 mM CaCl₂, 1.2 mM MgSO₄, 25 mM HEPES, 1 mM ascorbic acid, 0.02 mM pargyline HCl and 10 mM glucose, pH 7.4) and centrifuged for 15 min at 25,000×g. The final pellet was resuspended in perfusion buffer (1.4 mL) for immediate use.

The synaptosomal suspension was incubated for 10 min at 37° C. to restore metabolic activity. [³H]Dopamine ([³H]DA, specific activity=28.0 Ci/mmol, NEN Research Products) was added at a final concentration of 0.1 μM and the suspension was incubated at 37° C. for another 10 min. Aliquots of tissue (50 μL) and perfusion buffer (100 μL) were loaded into the suprafusion chambers of a Brandel Suprafusion System (series 2500, Gaithersburg, Md.). Perfusion buffer (room temperature) was pumped into the chambers at a rate of 1.5 mL/min for a wash period of 16 min. Test compound (10 μM) or nicotine (10 μM) was then applied in the perfusion stream for 48 sec. Fractions (24 sec each) were continuously collected from each chamber throughout the experiment to capture basal release and agonist-induced peak release and to re-establish the baseline after the agonist application. The perfusate was collected directly into scintillation vials, to which scintillation fluid was added. [³H]DA released was quantified by scintillation counting. For each chamber, the integrated area of the peak was normalized to its baseline.

Release was expressed as a percentage of release obtained with an equal concentration of L-nicotine. Within each assay, each test compound was replicated using 2-3 chambers; replicates were averaged. When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also defined.

### Example 3

### Selectivity vs. Peripheral nAChRs

Interaction at the Human Muscle nAChR Subtype

Activation of muscle-type nAChRs was established on the human clonal line TE671/RD, which is derived from an embryonal rhabdomyosarcoma (Stratton et al., Carcinogen 10: 899 (1989)). These cells express receptors that have pharmacological (Lukas, J. Pharmacol. Exp. Ther. 251: 175 (1989)), electrophysiological (Oswald et al., Neurosci. Lett. 96: 207 (1989)), and molecular biological profiles (Luther et al., J. Neurosci. 9: 1082 (1989)) similar to the muscle-type nAChR.

TE671/RD cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). Cells were cultured in Dulbecco's modified Eagle's medium (Gibco/BRL) with 10% horse serum (Gibco/BRL), 5% fetal bovine serum (HyClone, Logan Utah), 1 mM sodium pyruvate, 4 mM L-Glutamine, and 50,000 units penicillin-streptomycin (Irvine Scientific). When cells were 80% confluent, they were plated to 12 well polystyrene plates (Costar). Experiments were conducted when the cells reached 100% confluency.

Nicotinic acetylcholine receptor (nAChR) function was assayed using ⁸⁶Rb⁺ efflux according to the method described by Lukas et al., Anal. Biochem. 175: 212 (1988). On the day of the experiment, growth media was gently removed from the well and growth media containing ⁸⁶Rubidium chloride (10⁶ μCi/mL) was added to each well. Cells were incubated at 37° C. for a minimum of 3 h. After the loading period, excess ⁸⁶Rb⁺ was removed and the cells were washed twice with label-free Dulbecco's phosphate buffered saline (138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH. 7.4), taking care not to disturb the cells. Next, cells were exposed to either 100 μM of test compound, 100 μM of L-nicotine (Acros Organics) or buffer alone for 4 min. Following the exposure period, the supernatant containing the released ⁸⁶Rb⁺ was removed and transferred to scintillation vials. Scintillation fluid was added and released radioactivity was measured by liquid scintillation counting.

Within each assay, each point had 2 replicates, which were averaged. The amount of ⁸⁶Rb⁺ release was compared to both a positive control (100 μM L-nicotine) and a negative control (buffer alone) to determine the percent release relative to that of L-nicotine.

When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also determined.

Interaction at the Rat Ganglionic nAChR Subtype

Activation of rat ganglion nAChRs was established on the pheochromocytoma clonal line PC12, which is a continuous clonal cell line of neural crest origin, derived from a tumor of the rat adrenal medulla. These cells express ganglion-like nAChRs (see Whiting et al., Nature 327: 515 (1987); Lukas, J. Pharmacol. Exp. Ther. 251: 175 (1989); Whiting et al., Mol. Brain. Res. 10: 61 (1990)).

Rat PC12 cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). Cells were cultured in Dulbecco's modified Eagle's medium (Gibco/BRL) with 10% horse serum (Gibco/BRL), 5% fetal bovine serum (HyClone, Logan Utah), 1 mM sodium pyruvate, 4 mM L-Glutamine, and 50,000 units penicillin-streptomycin (Irvine Scientific). When cells were 80% confluent, they were plated to 12 well Nunc plates (Nunclon) and coated with 0.03% poly-L-lysine (Sigma, dissolved in 100 mM boric acid). Experiments were conducted when the cells reached 80% confluency.

Nicotinic acetylcholine receptor (nAChR) function was assayed using ⁸⁶Rb⁺ efflux according to a method described by Lukas et al., Anal. Biochem. 175: 212 (1988). On the day of the experiment, growth media was gently removed from the well and growth media containing ⁸⁶Rubidium chloride (10⁶ μCi/mL) was added to each well. Cells were incubated at 37° C. for a minimum of 3 h. After the loading period, excess ⁸⁶Rb⁺ was removed and the cells were washed twice with label-free Dulbecco's phosphate buffered saline (138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH. 7.4), taking care not to disturb the cells. Next, cells were exposed to either 100 μM of test compound, 100 μM of nicotine or buffer alone for 4 min. Following the exposure period, the supernatant containing the released ⁸⁶Rb⁺ was removed and transferred to scintillation vials. Scintillation fluid was added and released radioactivity was measured by liquid scintillation counting

Within each assay, each point had 2 replicates, which were averaged. The amount of ⁸⁶Rb⁺ release was compared to both a positive control (100 μM nicotine) and a negative control (buffer alone) to determine the percent release relative to that of L-nicotine.

When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also determined.

Interaction at the Human Ganglionic nAChR Subtype

The cell line SH-SY5Y is a continuous line derived by sequential subcloning of the parental cell line, SK-N-SH, which was originally obtained from a human peripheral neuroblastoma. SH-SY5Y cells express a ganglion-like nAChR (Lukas et al., Mol. Cell. Neurosci. 4: 1 (1993)).

Human SH-SY5Y cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). Cells were cultured in Dulbecco's modified Eagle's medium (Gibco/BRL) with 10% horse serum (Gibco/BRL), 5% fetal bovine serum (HyClone, Logan Utah), 1 mM sodium pyruvate, 4 mM L-Glutamine, and 50,000 units penicillin-streptomycin (Irvine Scientific). When cells were 80% confluent, they were plated to 12 well polystyrene plates (Costar). Experiments were conducted when the cells reached 100% confluency.

Nicotinic acetylcholine receptor (nAChR) function was assayed using ⁸⁶Rb⁺ efflux according to a method described by Lukas et al., Anal. Biochem. 175: 212 (1988). On the day of the experiment, growth media was gently removed from the well and growth media containing ⁸⁶Rubidium chloride (10⁶ μCi/mL) was added to each well. Cells were incubated at 37° C. for a minimum of 3 h. After the loading period, excess ⁸⁶Rb⁺ was removed and the cells were washed twice with label-free Dulbecco's phosphate buffered saline (138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH 7.4), taking care not to disturb the cells. Next, cells were exposed to either 100 μM of test compound, 100 μM of nicotine, or buffer alone for 4 min. Following the exposure period, the supernatant containing the released ⁸⁶Rb⁺ was removed and transferred to scintillation vials. Scintillation fluid was added and released radioactivity was measured by liquid scintillation counting

Within each assay, each point had 2 replicates, which were averaged. The amount of ⁸⁶Rb⁺ release was compared to both a positive control (100 μM nicotine) and a negative control (buffer alone) to determine the percent release relative to that of L-nicotine.

When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also defined.

### Example 4

### Novel Object Recognition (NOR) Task

The novel object recognition (NOR) task was performed in accord with the description of Ennaceur and Delacour Behav. Brain Res. 100: 85-92 (1988).

### Synthetic Examples

### Example 5

### Synthesis of N-(tert-butoxycarbonyl)-3,7-diazabicyclo[3.3.0]octane

N-(tert-Butoxycarbonyl)-3,7-diazabicyclo[3.3.0]octane was prepared as described in U.S. applications 2004/0186107 to Schrimpf et al. and 2005/0101602 to Basha et al., according to the following procedures:

### 5-Benzyltetrahydropyrrolo[3,4-c]pyrrole-1,3-dione (or 7-benzyl-3,7-diazabicyclo[3.3.0]octan-2,4-dione)

Trifluoroacetic acid (TFA, 0.50 mL, 6.5 mmol) was added to a cold (0° C.) solution of maleimide (6.27 g, 0.0646 mol) in dichloromethane (150 mL) under nitrogen. A solution of N-(methoxymethyl)-N-(trimethylsilylmethyl)benzylamine (20 g, 0.084 mol) in dichloromethane (100 mL) was added drop-wise over 45 min. After the addition was complete, the mixture was warmed slowly to ambient temperature and stirred for 16 h. The mixture was concentrated and the resulting residue was dissolved in dichloromethane (200 mL) and washed with saturated aqueous sodium bicarbonate (2×50 mL). The aqueous layer was separated and extracted with dichloromethane (2×75 mL). The combined dichloromethane extracts were washed with brine (50 mL), dried over anhydrous magnesium sulfate, filtered and concentrated to give 12.5 g (83.9% yield) of a light yellow, waxy solid (MS m/z 231 (M+H)).

### 2-Benzyloctahydropyrrolo[3,4-c]pyrrole (or 3-benzyl-3,7-diazabicyclo[3.3.0]octane)

The crude 5-benzyltetrahydropyrrolo[3,4-c]pyrrole-1,3-dione (4.9 g, 0.021 mol) was dissolved in cold (0° C.) dry tetrahydrofuran (THF) (50 mL) under nitrogen, and lithium aluminum hydride (63 mL of 1 M in THF, 0.063 mol) was added drop-wise over 30 min to the continuously cooled solution. The resulting mixture was stirred at ambient temperature for 30 min and then warmed to reflux for 4 h. The mixture was then cooled to 0° C. and quenched by the slow addition of excess solid sodium sulfate decahydrate. The mixture was warmed to ambient temperature and stirred for 16 h. The solids were filtered and the residue was washed with ethyl acetate (3×100 mL). The combined filtrates were concentrated to give 4.2 g (99% yield) of a waxy solid (MS m/z 203 (M+H)).

### 5-Benzylhexahydropyrrolo[3,4-c]pyrrole-2-carboxylic acid tert-butyl ester (or N-benzyl-N′-(tert-butoxycarbonyl)-3,7-diazabicyclo[3.3.0]octane)

The crude 2-benzyloctahydropyrrolo[3,4-c]pyrrole (4.2 g, 0.021 mol) was dissolved in THF (50 mL). Di-t-butyl dicarbonate (5.5 g, 0.025 mol) and aqueous saturated sodium bicarbonate (10 mL) were added, and the mixture was stirred at ambient temperature overnight. The reaction was quenched with water (10 mL), and ethyl acetate (30 mL) was added. The aqueous layer was extracted with ethyl acetate (2×20 mL), and the combined organic extracts were dried over anhydrous sodium sulfate and concentrated. Purification via silica gel column chromatography (1:1 hexanes) ethyl acetate) gave 5.07 g (79.8% yield) of the title compound (MS m/z 303 (M+H)).

### Hexahydropyrrolo[3,4-c]pyrrole-2-carboxylic acid tert-butyl ester (or N-(tert-butoxycarbonyl)-3,7-diazabicyclo[3.3.0]octane)

The 5-benzylhexahydropyrrolo[3,4-c]pyrrole-2-carboxylic acid tert-butyl ester (5.07 g, 0.0168 mol) was dissolved in methanol (50 mL) and 20% Pd(OH)₂/C (wet) (˜2 g) was added under a nitrogen atmosphere. The resulting mixture was warmed (45-50° C.) and shaken for 2 h under 40 psi of hydrogen. The mixture was filtered and concentrated to give 3.49 g (97.7% yield) of the title compound (MS m/z 213 (M+H)).

### Example 6

### Synthesis of 3,7-diazabicyclo[3.3.1]nonane-3-carboxylic acid tert-butyl ester

3,7-Diazabicyclo[3.3.1]nonane-3-carboxylic acid tert-butyl ester (or N-(tert-butoxycarbonyl)-3,7-diazabicyclo[3.3.1]nonane) was prepared according to the following procedures:

### 7-Benzyl-3,7-diazabicyclo[3.3.1]nonane-3-carboxylic acid tert-butyl ester (or N-benzyl-N′-(tert-butoxycarbonyl)-3,7-diazabicyclo[3.3.1]nonane)

7-Benzyl-3,7-diazabicyclo[3.3.1]nonane-3-carboxylic acid tert-butyl ester was prepared according to procedures set forth by Stead et al. in Org. Lett. 7(20): 4459 (2005).

### 3,7-Diazabicyclo[3.3.1]-3-carboxylic acid tert-butyl ester

7-Benzyl-3,7-diazabicyclo[3.3.1]nonane-3-carboxylic acid tert-butyl ester (0.49 g, 1.6 mmol) was dissolved in methanol (20 mL) and 20% Pd(OH)₂/C (wet) (˜2 g) was added under a nitrogen atmosphere. This mixture was warmed to about 50° C. and shaken for 2 h under 55 psi of hydrogen. The resulting mixture was filtered and concentrated to give 0.32 g (94% yield) of the title compound (MS m/z 227 (M+H)).

### Example 7

### Synthesis of N-(propanoyl)-3,7-diazabicyclo[3.3.0]octane

1-(Hexahydropyrrolo[3,4-c]pyrrol-2-yl)-1-propanone hemigalactarate (or N-(propanoyl)-3,7-diazabicyclo[3.3.0]octane hemigalactarate), was prepared according to the following techniques, illustrative of the coupling reaction used to make aliphatic amides of 3,7-diazabicyclo[3.3.0]octane and 3,7-diazabicyclo[3.3.1]nonane:

### 1-(Hexahydropyrrolo[3,4-c]pyrrol-2-yl)-1-propanone hemigalactarate (or N-(propanoyl)-3,7-diazabicyclo[3.3.0]octane hemigalactarate)

To a mixture of hexahydropyrrolo[3,4-c]pyrrole-2-carboxylic acid tert-butyl ester (1.4 g, 6.6 mmol), propionic acid (0.49 mL, 6.6 mmol) and triethylamine (2.8 mL, 20 mmol) in dichloromethane (50 mL) was added O-(benzotriazol-1-yl)-N,N,N′,N′-tetramethyluronium hexafluorophosphate (HBTU) (2.5 g, 6.6 mmol), and the mixture was stirred for 18 h at ambient temperature. The reaction was diluted with chloroform (50 mL), washed sequentially with water (2×50 mL) and 20% aqueous potassium carbonate (2×50 mL), and dried over anhydrous magnesium sulfate. The volatiles were evaporated, and the crude product was purified by HPLC, using acetonitrile and 0.05% aqueous trifluoroacetic acid (TFA) as mobile phase, to give 5-propanoylhexahydropyrrolo[3,4-c]pyrrole-2-carboxylic acid tert-butyl ester. This was treated with 4 M hydrochloric acid in 1,4-dioxane (10 mL) for 16 h at ambient temperature. The supernatant liquid was decanted and white precipitate was washed with ether (10 mL) and dried in vacuo. It was then dissolved in water (20 mL), treated with Amberlyst A26 (3 g) and filtered. The filtrate was concentrated to give 0.75 g (4.4 mmol) of free base as light yellow oil. A suspension of galactaric (mucic) acid (0.47 g, 2.2 mmol) in ethanol (10 mL) was added. The mixture was heated and stirred as water was added drop-wise until the mixture became clear. The solution was filtered while still hot, and filtrate was kept at ambient temperature for 2 h. The precipitate was collected by vacuum filtration and dried to obtain 1-(hexahydropyrrolo[3,4-c]pyrrol-2-yl)-1-propanone hemigalactarate (0.44 g) as white crystals. ¹H NMR (D₂O, 300 MHz): δ 4.11 (s, 1H, galactaric acid), 3.80 (s, 1H, galactaric acid), 3.72-3.65 (m, 1H), 3.59-3.42 (m, 4H), 3.37-3.28 (m, 1H), 3.12-3.05 (m, 4H), 2.23 (q, J=7.5 Hz, 2H), 0.95 (t, J=7.5 Hz, 3H); MS (m/z): 169 (M+1).

### Example 8

### Synthesis of N-(2,2,3,3-tetramethylcyclopropylcarbonyl)-3,7-diazabicyclo[3.3.0]octane

(Hexahydropyrrolo[3,4-c]pyrrol-2-yl)(2,2,3,3-tetramethylcyclopropyl)methanone hydrochloride or (N-(2,2,3,3-tetramethylcyclopropylcarbonyl)-3,7-diazabicyclo[3.3.0]octane hydrochloride) was prepared according to the following procedures and is illustrative of the coupling reaction used to make aliphatic amides of 3,7-diazabicyclo[3.3.0]octane and 3,7-diazabicyclo[3.3.1]nonane:

### (Hexahydropyrrolo[3,4-c]pyrrol-2-yl)(2,2,3,3-tetramethylcyclopropyl)methanone hydrochloride or N-(2,2,3,3-tetramethylcyclopropylcarbonyl)-3,7-diazabicyclo[3.3.0]octane hydrochloride

To a mixture of hexahydropyrrolo[3,4-c]pyrrole-2-carboxylic acid tert-butyl ester (1.00 g, 4.72 mmol), 2,2,3,3-tetramethylcyclopropanecarboxylic acid (0.80 g, 5.7 mmol) and triethylamine (2.8 mL, 20 mmol) in dichloromethane (50 mL) was added O-(benzotriazol-1-yl)-N,N,N′,N′-tetramethyluronium hexafluorophosphate (HBTU) (2.15 g, 5.66 mmol), and the mixture was stirred for 18 h at ambient temperature. The reaction was diluted with chloroform (50 mL), washed sequentially with water (2×50 mL) and 20% aqueous potassium carbonate (2×50 mL), and dried over anhydrous magnesium sulfate. The volatiles were evaporated, and the crude product was purified on HPLC, using acetonitrile and 0.05% aqueous TFA as mobile phase, to give 5-(2,2,3,3-tetramethyl-cyclopropanecarbonyl)hexahydropyrrolo[3,4-c]pyrrole-2-carboxylic acid tert-butyl ester. This was treated with 4 M hydrochloric acid in 1,4-dioxane (10 mL) for 16 h at ambient temperature. The precipitate was collected by vacuum filtration, washed with ethyl acetate (10 mL) and dried to obtain hexahydropyrrolo[3,4-c]pyrrol-2-yl)(2,2,3,3-tetramethylcyclopropyl)methanone hydrochloride (0.38 g) as white powder. ¹H NMR (D₂O, 300 MHz): δ 3.78-3.72 (m, 1H), 3.61-3.46 (m, 4H), 3.37-3.31 (m, 1H), 3.10-3.07 (m, 4H), 1.16 (s, 1H), 1.07 (s, 6H), 1.02 (s, 3H), 1.00 (s, 3H); MS (m/z): 237 (M+1).

### Example 9

### Synthesis of N-(cis-2-fluorocyclopropylcarbonyl)-3,7-diazabicyclo[3.3.0]octane

(Cis-2-fluorocyclopropyl)(hexahydro-pyrrolo[3,4-c]pyrrol-2-yl)methanone trifluoroacetate (or N-(cis-2-fluorocyclopropylcarbonyl)-3,7-diazabicyclo[3.3.0]octane trifluoroacetate), was prepared according to the following procedures and is illustrative of the coupling reaction used to make aliphatic amides of 3,7-diazabicyclo[3.3.0]octane and 3,7-diazabicyclo[3.3.1]nonane:

### (Cis-2-fluorocyclopropyl)(hexahydro-pyrrolo[3,4-c]pyrrol-2-yl)methanone trifluoroacetate or N-(cis-2-fluorocyclopropylcarbonyl)-3,7-diazabicyclo[3.3.0]octane trifluoroacetate

To a mixture of hexahydropyrrolo[3,4-c]pyrrole-2-carboxylic acid tert-butyl ester (0.060 g, 0.28 mmol), cis-2-fluorocyclopropanecarboxylic acid (0.035 g, 0.34 mmol) and triethylamine (0.195 mL, 1.4 mmol) in acetonitrile (10 mL) was added O-(benzotriazol-1-yl)-N,N,N′,N′-tetramethyluronium hexafluorophosphate (HBTU) (0.13 g, 0.34 mmol), and the mixture was stirred for 18 h at ambient temperature. The reaction was diluted with ethyl acetate (30 mL), washed with 10% aqueous sodium bicarbonate solution (2×20 mL), and dried over anhydrous magnesium sulfate. The volatiles were evaporated, and the crude product was purified on HPLC, using acetonitrile and 0.05% aqueous trifluoroacetic acid (TFA) as mobile phase to give 5-(2-fluoro-cyclopropanecarbonyl)-hexahydro-pyrrolo[3,4-c]pyrrole-2-carboxylic acid tert-butyl ester. This was treated 50% TFA in dichloromethane (5 mL) for 2 h at ambient temperature. Solvent was evaporated and product was purified on HPLC using acetonitrile and 0.05 aqueous TFA to obtain (2-fluorocyclopropyl)(hexahydro-pyrrolo[3,4-c]pyrrol-2-yl)methanone trifluoroacetate (0.020 g) as an oil. ¹H NMR (CD₃OD, 300 MHz): δ 5.00-4.96 and 4.78-4.72 (m, 1H), 3.96-3.89 (m, 1H), 3.79-3.53 (m, 5H), 3.25-3.04 (m, 4H), 2.07-1.97 (m, 1H), 1.74-1.62 (m, 1H), 1.18-1.04 (m, 1H); MS (m/z): 199 (M+1)).

As will be appreciated by those skilled in the art, analogous procedures to those described hereinabove as Examples 8 and 9 may be used to make the counterpart [3.3.1]nonane derivatives using the 3,7-diazabicyclo[3.3.1]nonane-3-carboxylic acid tert-butyl ester as herein described.

### Example 10

### Tabular Spectral and Receptor Binding Data

The above illustrated amide coupling procedures were used as a basis to make the compounds shown in Tables 1 and 2. Reagents and conditions will be readily apparent to those skilled in the art. In some cases, compounds were characterized by nuclear magnetic resonance (NMR) data. In other cases, compounds were structurally characterized by LCMS only.

TABLE 1

| | | Rat α4β2 | Human | |
|---|---|---|---|---|
| Example | Structure | Ki | α4β2 Ki | α7 Ki |
| 10-1 | 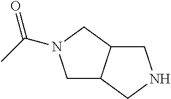 | 20.5 | 38.5 | ND; failed HTS |
| 10-2 | 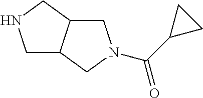 | 17.3 | 1.8 | 918.7 |
| 10-3 | 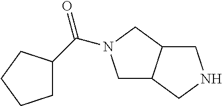 | 27.7 | 29.6 | ND; failed HTS |
| 10-4 | 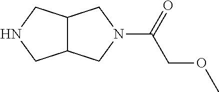 | 136.6 | 322.7 | 12360.4 |
| 10-5 | 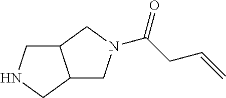 | 22.5 | 140.5 | ND; failed HTS |
| 10-6 | 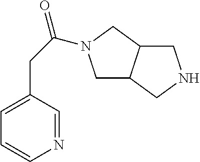 | ND; failed HTS | 489.5 | ND; failed HTS |
| 10-7 | 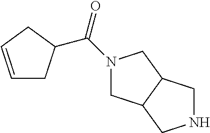 | 28.4 | 72.2 | ND; failed HTS |
| 10-8 | 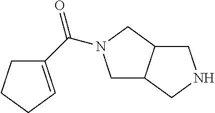 | 116 | 296.5 | ND; failed HTS |
| 10-9 | 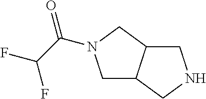 | 4.3 | 31.7 | ND; failed HTS |
| 10-10 | 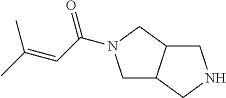 | 54.2 | 79.7 | ND; failed HTS |
| 10-11 | 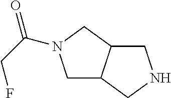 | 62.2 | 45.1 | ND; failed HTS |
| 10-12 | 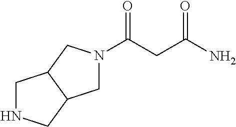 | 1390.8 | 745.8 | ND; failed HTS |
| 10-13 | 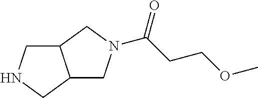 | 305.3 | 219.9 | ND; failed HTS |
| 10-14 | 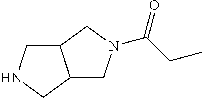 | 16.9 | 8.5 | ND; failed HTS |
| 10-15 | 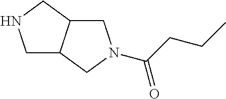 | 111.7 | 48.8 | ND; failed HTS |
| 10-16 | 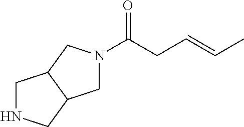 | 66 | 37.5 | ND; failed HTS |
| 10-17 | 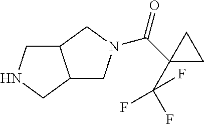 | 419.1 | 302.6 | ND; failed HTS |
| 10-18 | 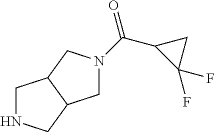 | 6.2 | 2.1 | ND; failed HTS |
| 10-19 | 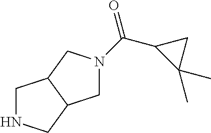 | 7.7 | 16.9 | ND; failed HTS |
| 10-20 | 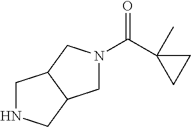 | 136.5 | 53.9 | ND; failed HTS |
| 10-21 | 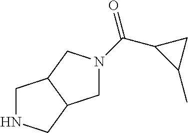 | 4.1 | 4.2 | ND; failed HTS |
| 10-22 | 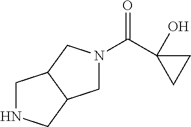 | 445.4 | 343 | ND; failed HTS |
| 10-23 | 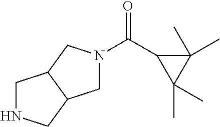 | 49.1 | 16.4 | ND; failed HTS |
| 10-24 | 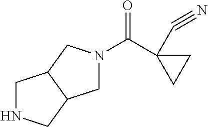 | 572.1 | 273.7 | ND; failed HTS |
| 10-25 | 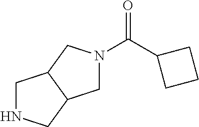 | 12.5 | 5.8 | ND; failed HTS |
| 10-26 | 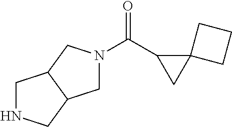 | 6.3 | 4 | ND; failed HTS |
| 10-27 | 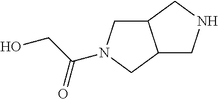 | 264.4 | 202.3 | ND; failed HTS |
| 10-28 | 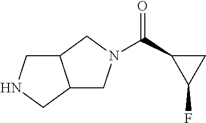 | 10 | 2.9 | ND; failed HTS |
| 10-29 | 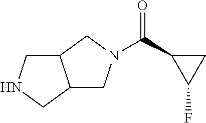 | 21.3 | 5.9 | ND; failed HTS |
| 10-30 | 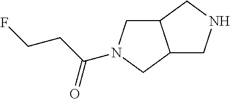 | 22.4 | 14.8 | ND; failed HTS |
| 10-31 | 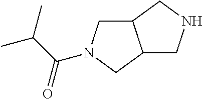 | 56 | 28.6 | ND; failed HTS |
| 10-32 | 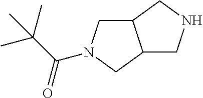 | 15.8 | 8.1 | ND; failed HTS |
| 10-33 | 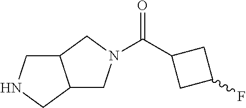 | 7.2 | 18 | ND; failed HTS |
| 10-34 | 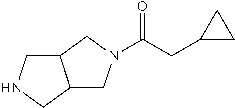 | 62.7 | 104.9 | ND; failed HTS |
| 10-35 | 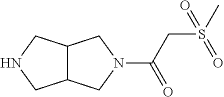 | 237.7 | 344.5 | ND; failed HTS |
| 10-36 | 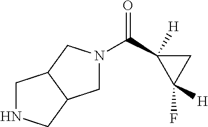 | 34.3 | 17.2 | 25328.9 |
| 10-37 | 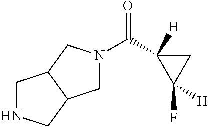 | 12.2 | 4.9 | ND; failed HTS |
| 10-38 | 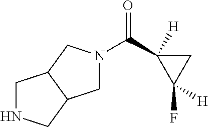 | 9.7 | 30 | ND; failed HTS |
| 10-39 | 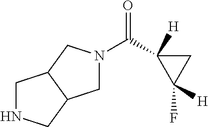 | 6.6 | 3.8 | ND; failed HTS |
| 10-40 | 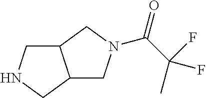 | 8.6 | 9.8 | ND; failed HTS |
| 10-41 | 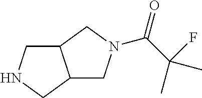 | 12 | 26.9 | ND; failed HTS |
| 10-42 | 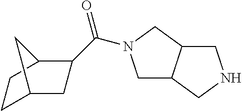 | 139 | 64.6 | ND; failed HTS |
| 10-43 | 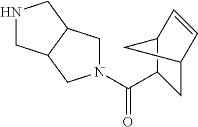 | 34 | 15.8 | ND; failed HTS |
| 10-44 | 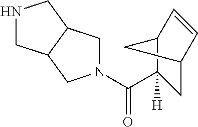 | 9.3 | 3.4 | ND; failed HTS |
| 10-45 | 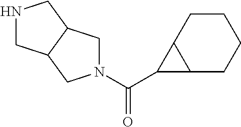 | 26 | 8.6 | 508.4 |
| 10-46 | 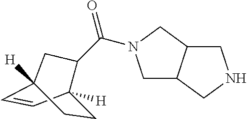 | 236.1 | 44.5 | ND; failed HTS |
| 10-47 | 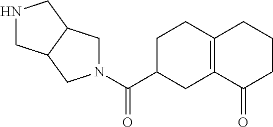 | 369.1 | 418.8 | ND; failed HTS |
| 10-48 | 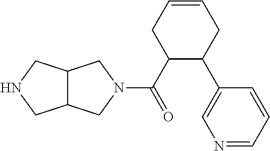 | 999.2 | 345.3 | ND; failed HTS |
| 10-49 | 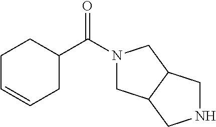 | 236.1 | 133 | ND; failed HTS |
| 10-50 | 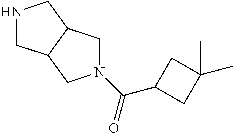 | ND; failed HTS | 128.6 | ND; failed HTS |
| 10-51 | 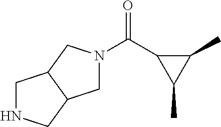 | 17.6 | 44.6 | 4789.4 |
| 10-52 | 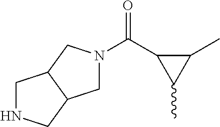 | 133.1 | 12.2 | ND; failed HTS |
| 10-53 | 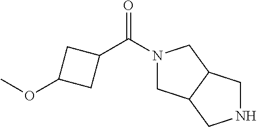 | 83.7 | 147.3 | ND; failed HTS |
| 10-54 | 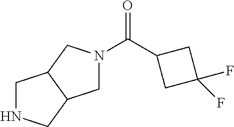 | 20.3 | 5.7 | ND; failed HTS |

TABLE 2

| | | | Human α4β2 | |
|---|---|---|---|---|
| Example | Structure | Rat α4β2 Ki | Ki | α7 Ki |
| 10-55 | 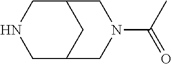 | 18.7 | 20.2 | 3277.9 |
| 10-56 | 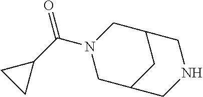 | 2.8 | 0.4 | 95.7 |
| 10-57 | 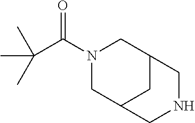 | 112 | 125.6 | ND; failed HTS |
| 10-58 | 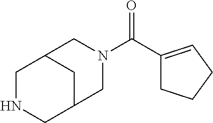 | 176.7 | 468.5 | ND; failed HTS |
| 10-59 | 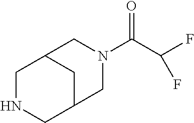 | 1.7 | 24.6 | 294.6 |
| 10-60 | 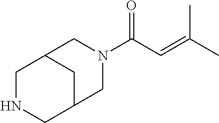 | 25.9 | 13.8 | ND; failed HTS |
| 10-61 | 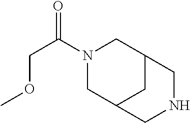 | 50 | 38.8 | 1472.9 |
| 10-62 | 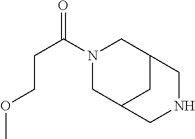 | 175 | 126.6 | ND; failed HTS |
| 10-63 | 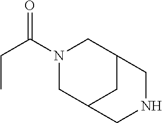 | 12.1 | 10.4 | 795.1 |
| 10-64 | 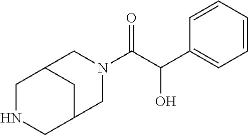 | ND; failed HTS | 1525.9 | ND; failed HTS |
| 10-65 | 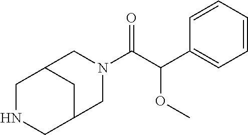 | ND; failed HTS | 385.6 | ND; failed HTS |
| 10-66 | 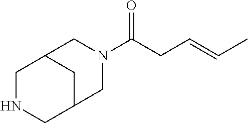 | 30.1 | 16.7 | ND; failed HTS |
| 10-67 | 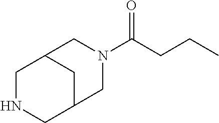 | 8.1 | 2.9 | 230.3 |
| 10-68 | 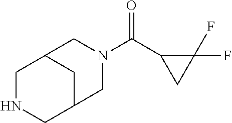 | 1.2 | 1.1 | 142.5 |
| 10-69 | 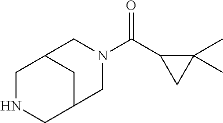 | 1.5 | 1 | 376.6 |
| 10-70 | 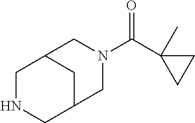 | 873.7 | 213.5 | ND; failed HTS |
| 10-71 | 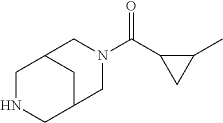 | 1.4 | 1 | 78.7 |
| 10-72 | 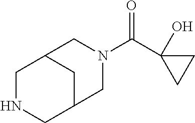 | 75.9 | 225.2 | ND; failed HTS |
| 10-73 | 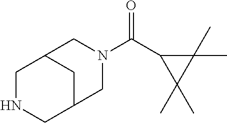 | 119.2 | 28.2 | ND; failed HTS |
| 10-74 | 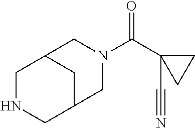 | 4842.2 | 1341.4 | ND; failed HTS |
| 10-75 | 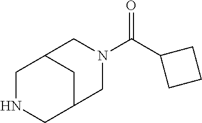 | 1.6 | 1.4 | 70 |
| 10-76 | 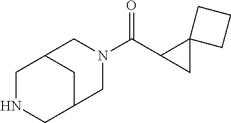 | 1.6 | 1.1 | 680.3 |
| 10-77 | 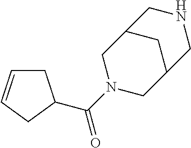 | 25.6 | 12.5 | 54 |
| 10-78 | 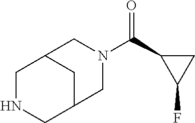 | 2.9 | 1.7 | 396.6 |
| 10-79 | 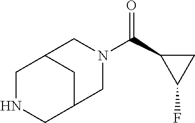 | 4.1 | 2 | 960.8 |
| 10-80 | 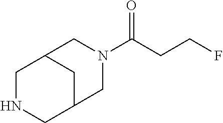 | 13.8 | 9.5 | ND; failed HTS |
| 10-81 | 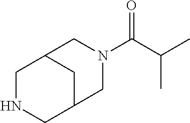 | 101.6 | 44.4 | 644.7 |
| 10-82 | 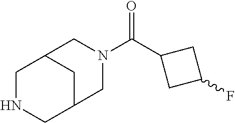 | 1.4 | 2.9 | 1043 |
| 10-83 | 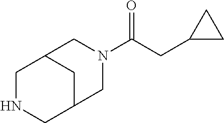 | 13.3 | 33.9 | ND; failed HTS |
| 10-84 | 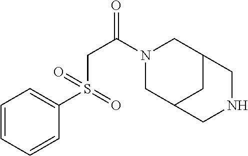 | 958.5 | 598.4 | ND; failed HTS |
| 10-85 | 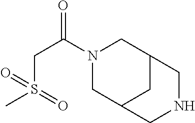 | 189.6 | 131.2 | ND; failed HTS |
| 10-86 | 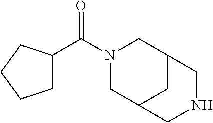 | 26.6 | 20.2 | 211.2 |
| 10-87 | 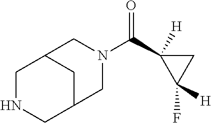 | 11.4 | 5.2 | 591.7 |
| 10-88 | 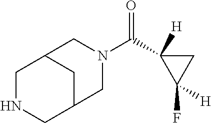 | 1.8 | 1.4 | 242.6 |
| 10-89 | 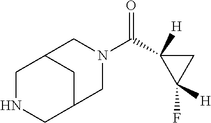 | 0.8 | 1 | 73.4 |
| 10-90 | 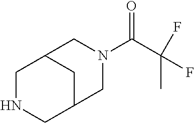 | 14.6 | 9.2 | ND; failed HTS |
| 10-91 | 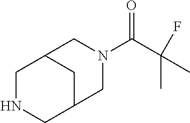 | 341.9 | 139.4 | ND; failed HTS |
| 10-92 | 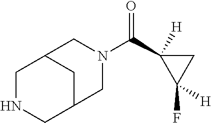 | 15.9 | 7.3 | 359.8 |
| 10-93 | 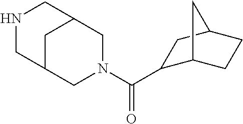 | 49.7 | 35.3 | ND; failed HTS |
| 10-94 | 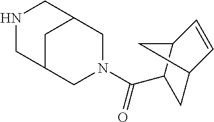 | 22.7 | 12.6 | ND; failed HTS |
| 10-95 | 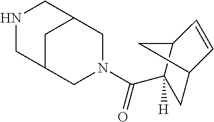 | 4.9 | 2.2 | ND; failed HTS |
| 10-96 | 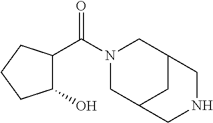 | 775.5 | 730.6 | ND; failed HTS |
| 10-97 | 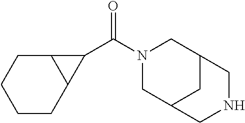 | ND; failed HTS | 64.1 | ND; failed HTS |
| 10-98 | 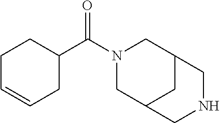 | 197.8 | 133.7 | 794.2 |
| 10-99 | 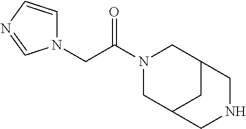 | 1320.6 | 821 | ND; failed HTS |
| 10-100 | 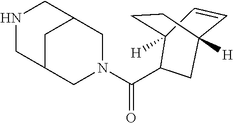 | 255.6 | 72.4 | ND; failed HTS |
| 10-101 | 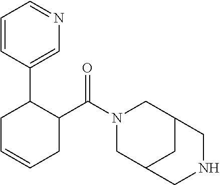 | 602.1 | 69.6 | ND; failed HTS |

### Summary of Biological Data

Compounds of Tables 1 and 2, representative of the present invention, exhibited inhibition constants (Ki values) at the rat and human α4β2 subtypes in the ranges of 1 nM to 5000 nM and 1 nM to 1500 nM respectively, indicating high affinity for the α4β2 subtype. Ki values at the α7 subtype vary within the range of 50 nM to 12,000 nM, indicating lower affinity for the α7 subtype. Furthermore, some compounds failed to bind sufficiently in high through-put screening (HTS) to warrant Ki determination. This was much more common for binding at the α7 subtype, as compared to the α4β2 subtype.

In this regard, the notation “failed HTS” as used herein for α4β2 subtype binding means that the compound failed to inhibit, at 5 μM concentration, the binding of 5 nM ³H-nicotine by at least 50%. The notation “failed HIS” as used herein for α7 subtype binding means that the compound failed to inhibit, at 5 μM concentration, the binding of 5 nM ³H-MLA (methyllycaconitine) by at least 50%.

Certain exemplified compounds were assessed in the NOR task. Thus, N-(propanoyl)-3,7-diazabicyclo[3.3.0]octane (Compound 10-16, Table 1) was active in NOR in rats, at 0.1 mg/kg. This provides evidence of the efficacy (and potency) of the compounds of the present invention in treating cognitive deficits, attentional disorders and dementias, and the potential of these compounds for human therapy.

Test compounds were employed in free or salt form.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula I: 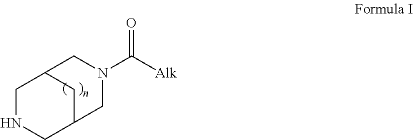
wherein n is 0 or 1;
Alk is alkyl, cycloalkyl, alkenyl, cycloalkenyl, alkynyl, or cycloalkynyl, each of which may be substituted with one, two, or three of alkyl, substituted alkyl, alkenyl, substituted alkenyl, alkynyl, substituted alkynyl, heterocyclyl, substituted heterocyclyl, cycloalkyl, substituted cycloalkyl, aryl, heteroaryl, substituted aryl, substituted heteroaryl, alkylaryl, alkylheteroaryl, substituted alkylaryl, substituted alkylheteroaryl, arylalkyl, heteroarylalkyl, substituted arylalkyl, substituted heteroarylalkyl, halogen, —OR′, ═O, —NR′R″, haloalkyl, —CN, —NO₂, —SR′, —N₃, —C(═O)NR′R″, —NR′C(═O)R″, —C(═O)R′, —C(═O)OR′, —OC(═O)R′, —OC(═O)NR′R″, —NR′C(═O)OR″, —SO₂R′, —SO₂NR′R″, and —NR′SO₂R″,
where R′ and R″ are independently selected from hydrogen, alkyl, cycloalkyl, heterocyclyl, aryl, heteroaryl, or arylalkyl,
or R′ and R″ and the atoms to which they are attached together can form a three- to eight-membered heterocyclic ring,
wherein the term “substituted”, as applied to alkyl, alkenyl, alkynyl, heterocyclyl, cycloalkyl, aryl, heteroaryl, alkylaryl, alkylheteroaryl, arylalkyl, and heteroarylalkyl, refers to substitution with one or more alkyl, aryl, heteroaryl, halogen, —OR′, or —NR′R″ groups, where R′ and R″ are as defined;
or a pharmaceutically acceptable salt thereof.

2. The compound according to claim 1, wherein:
n has the value of 0 or 1;
Alk is methyl, ethyl, n-propyl, isopropyl, 1-propenyl, allyl, n-butyl, 1-butenyl, 2-butenyl, 3-butenyl, isobutyl, sec-butyl, tert-butyl, cyclopropyl, cyclobutyl, cyclopentyl, cyclopentenyl, cyclohexyl, cyclohexenyl, spirobicyclohexyl, cycloheptyl, bicycloheptyl, bicycloheptenyl, cyclooctyl, bicyclooctyl, or bicyclooctenyl,
each of which may be substituted with one, two, or three of alkyl, aryl, heteroaryl, substituted aryl, substituted heteroaryl, halogen, —OR′, ═O, haloalkyl, —CN, —NO₂, —C≡CR′, —SR′, —N₃, —C(═O)NR′R″, —NR′C(═O)R″, —C(═O)R′, —C(═O)OR′, —OC(═O)R′, —OC(═O)NR′R″, —NR′C(═O)OR″, —SO₂R′, —SO₂NR′R″, or —NR′SO₂R″,
where R′ and R″ are defined as in claim 1,
wherein the term “substituted” is defined as in claim 1,
or a pharmaceutically acceptable salt thereof.

3. The pharmaceutically acceptable salts of claim 1, wherein Alk is methyl, ethyl or n-propyl.

4. A pharmaceutically acceptable salt of Formula Ia: 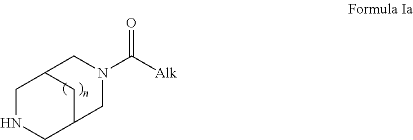
wherein n is 0 or 1; and
Alk is methyl, ethyl, or n-propyl.

5. The pharmaceutically acceptable salts of claim 1, wherein Alk cyclopropyl.

6. A pharmaceutically acceptable salt of Formula Ia: 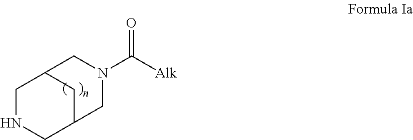
wherein n is 0 or 1; and
Alk is cyclopropyl substituted with one or more halogen.

7. The compound according to claim 1, wherein n is 0.

8. The compound according to claim 1, wherein n is 1.

9. A compound selected from the group consisting of:
N-(acetyl)-3,7-diazabicyclo[3.3.0]octane,
N-(fluoroacetyl)-3,7-diazabicyclo[3.3.0]octane,
N-(methoxyacetyl)-3,7-diazabicyclo[3.3.0]octane,
N-(2-phenyl-2-methoxyacetyl)-3,7-diazabicyclo[3.3.0]octane,
N-(hydroxyacetyl)-3,7-diazabicyclo[3.3.0]octane,
N-(difluoroacetyl)-3,7-diazabicyclo[3.3.0]octane,
N-(carbamoylacetyl)-3,7-diazabicyclo[3.3.0]octane,
N-(methylsulfonylacetyl)-3,7-diazabicyclo[3.3.0]octane,
N-(phenylsulfonylacetyl)-3,7-diazabicyclo[3.3.0]octane,
N-(cyclopropylacetyl)-3,7-diazabicyclo[3.3.0]octane,
N-(propanoyl)-3,7-diazabicyclo[3.3.0]octane,
N-(3-fluoropropanoyl)-3,7-diazabicyclo[3.3.0]octane,
N-(3-methoxypropanoyl)-3,7-diazabicyclo[3.3.0]octane,
N-(2,2-difluoropropanoyl)-3,7-diazabicyclo[3.3.0]octane,
N-(2-propenoyl)-3,7-diazabicyclo[3.3.0]octane,
N-(butanoyl)-3,7-diazabicyclo[3.3.0]octane,
N-(2-butenoyl)-3,7-diazabicyclo[3.3.0]octane,
N-(3-butenoyl)-3,7-diazabicyclo[3.3.0]octane,
N-(2-methylpropanoyl)-3,7-diazabicyclo[3.3.0]octane,
N-(2-fluoro-2-methylpropanoyl)-3,7-diazabicyclo[3.3.0]octane,
N-(pentanoyl)-3,7-diazabicyclo[3.3.0]octane,
N-(3-methylbutanoyl)-3,7-diazabicyclo[3.3.0]octane,
N-(2-methylbutanoyl)-3,7-diazabicyclo[3.3.0]octane,
N-(2,2-dimethylpropanoyl)-3,7-diazabicyclo[3.3.0]octane,
N-(3-methyl-2-butenoyl)-3,7-diazabicyclo[3.3.0]octane,
N-(3-pentenoyl)-3,7-diazabicyclo[3.3.0]octane,
N-(cyclopropylcarbonyl)-3,7-diazabicyclo[3.3.0]octane,
N-(2-fluorocyclopropylcarbonyl)-3,7-diazabicyclo[3.3.0]octane,
N-(1-methylcyclopropylcarbonyl)-3,7-diazabicyclo[3.3.0]octane,
N-(1-hydroxycyclopropylcarbonyl)-3,7-diazabicyclo[3.3.0]octane,
N-(1-cyanocyclopropylcarbonyl)-3,7-diazabicyclo[3.3.0]octane,
N-(2-methylcyclopropylcarbonyl)-3,7-diazabicyclo[3.3.0]octane,
N-(2,2-difluorocyclopropylcarbonyl)-3,7-diazabicyclo[3.3.0]octane,
N-(2,2-dimethylcyclopropylcarbonyl)-3,7-diazabicyclo[3.3.0]octane,
N-(2,2,3,3-tetramethylcyclopropylcarbonyl)-3,7-diazabicyclo[3.3.0]octane,
N-(cyclobutylcarbonyl)-3,7-diazabicyclo[3.3.0]octane,
N-(3-fluorocyclobutylcarbonyl)-3,7-diazabicyclo[3.3.0]octane,
N-(3,3-difluorocyclobutylcarbonyl)-3,7-diazabicyclo[3.3.0]octane,
N-(3,3-dimethylcyclobutylcarbonyl)-3,7-diazabicyclo[3.3.0]octane,
N-(3-methoxycyclobutylcarbonyl)-3,7-diazabicyclo[3.3.0]octane,
N-(cyclopentylcarbonyl)-3,7-diazabicyclo[3.3.0]octane,
N-(1-cyclopentenylcarbonyl)-3,7-diazabicyclo[3.3.0]octane,
N-(2-cyclopentenylcarbonyl)-3,7-diazabicyclo[3.3.0]octane,
N-(3-cyclopentenylcarbonyl)-3,7-diazabicyclo[3.3.0]octane,
N-(cyclohexylcarbonyl)-3,7-diazabicyclo[3.3.0]octane,
N-(3-cyclohexenylcarbonyl)-3,7-diazabicyclo[3.3.0]octane,
N-(norbornylcarbonyl)-3,7-diazabicyclo[3.3.0]octane,
N-(spiro[2.3]hexyl-1-carbonyl)-3,7-diazabicyclo[3.3.0]octane,
N-(bicyclo[4.1.0]heptyl-7-carbonyl)-3,7-diazabicyclo[3.3.0]octane,
N-(bicyclo[2.2.1]hept-5-enyl-2-carbonyl)-3,7-diazabicyclo[3.3.0]octane, and
N-(bicyclo[2.2.2]oct-5-enyl-2-carbonyl)-3,7-diazabicyclo[3.3.0]octane,
or a pharmaceutically acceptable salt thereof.

10. A compound selected from the group consisting of:
N-(acetyl)-3,7-diazabicyclo[3.3.1]nonane,
N-(fluoroacetyl)-3,7-diazabicyclo[3.3.1]nonane,
N-(methoxyacetyl)-3,7-diazabicyclo[3.3.1]nonane,
N-(2-phenyl-2-methoxyacetyl)-3,7-diazabicyclo[3.3.1]nonane,
N-(hydroxyacetyl)-3,7-diazabicyclo[3.3.1]nonane,
N-(difluoroacetyl)-3,7-diazabicyclo[3.3.1]nonane,
N-(carbamoylacetyl)-3,7-diazabicyclo[3.3.1]nonane,
N-(methylsulfonylacetyl)-3,7-diazabicyclo[3.3.1]nonane,
N-(phenylsulfonylacetyl)-3,7-diazabicyclo[3.3.1]nonane,
N-(cyclopropylacetyl)-3,7-diazabicyclo[3.3.1]nonane,
N-(propanoyl)-3,7-diazabicyclo[3.3.1]nonane,
N-(3-fluoropropanoyl)-3,7-diazabicyclo[3.3.1]nonane,
N-(3-methoxypropanoyl)-3,7-diazabicyclo[3.3.1]nonane,
N-(2,2-difluoropropanoyl)-3,7-diazabicyclo[3.3.1]nonane,
N-(2-propenoyl)-3,7-diazabicyclo[3.3.1]nonane,
N-(butanoyl)-3,7-diazabicyclo[3.3.1]nonane,
N-(2-butenoyl)-3,7-diazabicyclo[3.3.1]nonane,
N-(3-butenoyl)-3,7-diazabicyclo[3.3.1]nonane,
N-(2-methylpropanoyl)-3,7-diazabicyclo[3.3.1]nonane,
N-(2-fluoro-2-methylpropanoyl)-3,7-diazabicyclo[3.3.1]nonane,
N-(pentanoyl)-3,7-diazabicyclo[3.3.1]nonane,
N-(3-methylbutanoyl)-3,7-diazabicyclo[3.3.1]nonane,
N-(2-methylbutanoyl)-3,7-diazabicyclo[3.3.1]nonane,
N-(2,2-dimethylpropanoyl)-3,7-diazabicyclo[3.3.1]nonane,
N-(3-methyl-2-butenoyl)-3,7-diazabicyclo[3.3.1]nonane,
N-(3-pentenoyl)-3,7-diazabicyclo[3.3.1]nonane,
N-(cyclopropylcarbonyl)-3,7-diazabicyclo[3.3.1]nonane,
N-(2-fluorocyclopropylcarbonyl)-3,7-diazabicyclo[3.3.1]nonane,
N-(1-methylcyclopropylcarbonyl)-3,7-diazabicyclo[3.3.1]nonane,
N-(1-hydroxycyclopropylcarbonyl)-3,7-diazabicyclo[3.3.1]nonane,
N-(1-cyanocyclopropylcarbonyl)-3,7-diazabicyclo[3.3.1]nonane,
N-(2-methylcyclopropylcarbonyl)-3,7-diazabicyclo[3.3.1]nonane,
N-(2,2-difluorocyclopropylcarbonyl)-3,7-diazabicyclo[3.3.1]nonane,
N-(2,2-dimethylcyclopropylcarbonyl)-3,7-diazabicyclo[3.3.1]nonane,
N-(2,2,3,3-tetramethylcyclopropylcarbonyl)-3,7-diazabicyclo[3.3.1]nonane,
N-(cyclobutylcarbonyl)-3,7-diazabicyclo[3.3.1]nonane,
N-(3-fluorocyclobutylcarbonyl)-3,7-diazabicyclo[3.3.1]nonane,
N-(3,3-difluorocyclobutylcarbonyl)-3,7-diazabicyclo[3.3.1]nonane,
N-(3,3-dimethylcyclobutylcarbonyl)-3,7-diazabicyclo[3.3.1]nonane,
N-(3-methoxycyclobutylcarbonyl)-3,7-diazabicyclo[3.3.1]nonane,
N-(cyclopentylcarbonyl)-3,7-diazabicyclo[3.3.1]nonane,
N-(1-cyclopentenylcarbonyl)-3,7-diazabicyclo[3.3.1]nonane,
N-(2-cyclopentenylcarbonyl)-3,7-diazabicyclo[3.3.1]nonane,
N-(3-cyclopentenylcarbonyl)-3,7-diazabicyclo[3.3.1]nonane,
N-(cyclohexylcarbonyl)-3,7-diazabicyclo[3.3.1]nonane,
N-(3-cyclohexenylcarbonyl)-3,7-diazabicyclo[3.3.1]nonane,
N-(norbornylcarbonyl)-3,7-diazabicyclo[3.3.1]nonane,
N-(spiro[2.3]hexyl-1-carbonyl)-3,7-diazabicyclo[3.3.1]nonane,
N-(bicyclo[4.1.0]heptyl-7-carbonyl)-3,7-diazabicyclo[3.3.1]nonane,
N-(bicyclo[2.2.1]hept-5-enyl-2-carbonyl)-3,7-diazabicyclo[3.3.1]nonane, and
N-(bicyclo[2.2.2]oct-5-enyl-2-carbonyl)-3,7-diazabicyclo[3.3.1]nonane,
or a pharmaceutically acceptable salt thereof.

11. (canceled)

12. (canceled)

13. A method for treatment of a central nervous system disorder, comprising administering to a mammal in need of such treatment, a therapeutically effective amount of the compound according to claim 1.

14. The method of claim 13, wherein the disorder is selected from the group consisting of age-associated memory impairment, mild cognitive impairment, pre-senile dementia, early onset Alzheimer's disease, senile dementia, dementia of the Alzheimer's type, Lewy body dementia, vascular dementia, Alzheimer's disease, stroke, AIDS dementia complex, attention deficit disorder, attention deficit hyperactivity disorder, dyslexia, schizophrenia, schizophreniform disorder, schizoaffective disorder, cognitive deficits in schizophrenia, and cognitive dysfunction in schizophrenia.

15. The method of claim 13, wherein the disorder is selected from the group consisting of mild to moderate dementia of the Alzheimer's type, attention deficit disorder, attention deficit hyperactivity disorder, mild cognitive impairment, age-associated memory impairment, cognitive deficits in schizophrenia, and cognitive dysfunction in schizophrenia.

16. A pharmaceutical composition comprising a compound according to claim 1, and one or more pharmaceutically acceptable carrier.

17. (canceled)
